(19) United States
(12) Patent Application Publication
(10) Pub. No.: US 2025/0268985 A1
(43) Pub. Date: Aug. 28, 2025

(54) BCL2 INHIBITOR AND PEPTIDE COMBINATION THERAPY FOR TREATING PROLIFERATIVE DISEASES

(71) Applicant: IMMUNE SYSTEM KEY LTD., Jerusalem (IL)

(72) Inventor: Yoram DEVARY, Jerusalem (IL)

(73) Assignee: IMMUNE SYSTEM KEY LTD., Jerusalem (IL)

(21) Appl. No.: 18/856,098

(22) PCT Filed: Apr. 4, 2023
(86) PCT No.: PCT/IL2023/050371

§ 371 Date: Oct. 11, 2024

Related U.S. Application Data
(60) Provisional application No. 63/362,775, filed on Apr. 11, 2022.

(51) Int. Cl. A61K 38/22; A61K 31/404; A61K 31/4725; A61K 31/635; A61P 35/00; A61P 35/02

(52) U.S. Cl. CPC A61K 38/22; A61K 31/404; A61K 31/4725; A61K 31/635; A61P 35/00; A61P 35/02

(57) ABSTRACT

The present disclosure relates to a combinational therapy comprising Bcl2 inhibitors and peptides. The present disclosure further relates to compositions, methods and kits comprising Bcl2 inhibitors and peptides for the treatment of proliferative diseases.

### TECHNOLOGICAL FIELD

The present disclosure relates to combination therapy of B-cell lymphoma 2 (Bcl2) inhibitors with peptides for the treatment of proliferative diseases.

### BACKGROUND ART

References considered to be relevant as background to the presently disclosed subject matter are listed below:
[1] Lessene G, Czabotar P E, Colman P M. BCL-2 family antagonists for cancer therapy. Nat Rev Drug Discov. 2008; 7 (12): 989-1000.
[2] WO 2006/046239
[3] WO 2007/122622
[4] WO 2007/091240
[5] WO 2008/075349
[6] Sandler, U. et al., 2010, Recent advances in clinical medicine, ISSN: 1790-5125.
[7] Sandler, U. et al., 2010, J Experimental Therapeutics and Oncology 8:327-339.
[8] WO2015/083167
[9] WO2017/134668

Acknowledgement of the above references herein is not to be inferred as meaning that these are in any way relevant to the patentability of the presently disclosed subject matter.

### BACKGROUND OF THE INVENTION

BCL2-related proteins is a large family of proteins that operate by nonenzymatic protein: protein interactions to regulate the intrinsic or mitochondrial pathway to apoptosis, either protecting against apoptosis or promoting apoptosis.

The prosurvival proteins includes for example BCL-2 itself, B-cell lymphoma-extra large (BCL-xL), Myeloid Cell Leukemia-1 (MCL-1), B-cell lymphoma-w (BCL-w), and BCL-2-related gene expressed in fetal liver 1.

On the other hand, the proapoptotic proteins are further divided into (a) the proapoptotic BH3-only proteins, including BCL-2-interacting mediator of cell death (BIM), p53-upregulated modulator of apoptosis, and BCL-2-associated death promoter, and (b) the proapoptotic effector proteins BCL-2-associated X protein (BAX) and BCL-2 homologous antagonist killer (BAK).

The balance of activity between prosurvival proteins and BH3-only proapoptotic proteins determines whether a cell will live or undergo apoptosis, and collectively they serve to integrate the diverse extracellular and intracellular signals promoting either survival (e.g. growth factors, nutrients) or death induced by stress (e.g. oncogenic/proliferative, DNA damage, etc).

BH3-mimetics are a new class of anticancer drug that mimic the actions of BH3-only proteins in that they bind to prosurvival proteins like BCL2 in the same way and inhibit BCL2's ability to bind BAX or BAK [1]. As BCL2 also exists bound to native BH3-only proteins, BH3-mimetics can also displace these endogenous activators of apoptosis. For example, Venetoclax is a BCL2-selective BH3-mimetic and its addition to BCL2-overexpressing cancer cells in vitro potently triggers apoptosis

A peptide termed “T101” that is encoded by a cDNA unique for the human thymus was previously identified. This peptide as well as derivatives thereof were implicated, inter alia, for the treatment of cancer via the role of T101 as a stimulator of the immune system (WO 2006/046239, [2]). WO 2006/046239 demonstrates that T101 is able to stimulate the immune system and to reduce tumor size, suggesting that the peptide affects the proliferation of cancer cells. WO 2006/046239 also suggests an immune-based role for T101, for example in protecting patients during the course of standard chemotherapy.

Treatment of cancer by using T101 was also suggested in WO 2007/122622 [3], which demonstrates, inter alia, the effect of T101 on the development of various types of tumors. The peptide T101 was also described in WO 2007/091240 [4], relating to treatment of immunological diseases and in WO 2008/075349 [5] as well as in the publications by Sandler et al. [6-7], relating to treating or preventing a disease involving a cell having T1/ST2 receptor.

In addition, a peptide derivative of T101, termed “Nerofe”, has been reported to decrease the secretion of proteins that are known to be associated with cancer metastasis by cancer cells and to directly inhibit migration of cancer cells in vitro. In addition the peptide was shown to affect the serum level of vascular endothelial growth factor (VEGF) in cancer patients (WO2015/083167, [8]), and was suggested for use in a method of preventing or treating cancer metastasis.

Furthermore, the peptide termed “Nerofe” mentioned above was also shown in WO2017/134668 [9] to be implicated in the induction of ER stress which contributes to promoting cell death, and was suggested for reducing the administered standard of care doses of anti-cancer agents in treated cancer patients.

### SUMMARY OF THE INVENTION

In a first aspect, the present disclosure provides a combined composition comprising at least one inhibitor of a B-cell lymphoma 2 (Bcl2) prosurvival protein and an isolated peptide comprising the amino acid sequence denoted by SEQ ID NO. 1 or any functional derivative thereof, or pharmaceutically acceptable salt thereof and at least one pharmaceutically acceptable carrier, diluent, excipient and/or additive.

In a second aspect, the present disclosure provides a method of treating, preventing, ameliorating, reducing or delaying the onset of at least one proliferative disorder in a subject in need thereof, comprising the step of administering a therapeutically effective amount of at least one inhibitor of a Bcl2 prosurvival protein and an isolated peptide comprising the amino acid sequence denoted by SEQ ID NO. 1 or any functional derivative thereof, or pharmaceutically acceptable salt thereof, or any compositions, kits or combinations thereof.

In a third aspect, the present disclosure provides at least one inhibitor of a Bcl2 prosurvival protein and at least one isolated peptide comprising the amino acid sequence denoted by SEQ ID NO. 1 or any functional derivative thereof for use in a method of treating, preventing, ameliorating, reducing or delaying the onset of at least one proliferative disorder, or a pharmaceutically acceptable salt thereof.

In a fourth aspect, the present disclosure provides a kit comprising:
(a) at least one inhibitor of a Bcl2 prosurvival protein or any pharmaceutically acceptable salt thereof, optionally, in a first dosage form;
(b) an isolated peptide comprising the amino acid sequence denoted by SEQ ID NO. 1 or any functional derivative thereof, or pharmaceutically acceptable salt thereof, optionally, in a second dosage form.

In a fifth aspect, the present disclosure provides a method of treating, preventing, ameliorating, reducing or delaying the onset of at least one proliferative disorder in a subject treated with at least one inhibitor of a Bcl2 prosurvival protein, the method comprising the step of administering a therapeutically effective amount of and an isolated peptide comprising the amino acid sequence denoted by SEQ ID NO. 1 or any functional derivative thereof, or pharmaceutically acceptable salt thereof, or any compositions, kits or combinations thereof, to said subject.

In a sixth aspect, the present disclosure provides a method of treating, preventing, ameliorating, reducing or delaying the onset of at least one proliferative disorder in a subject treated with at least one isolated peptide comprising the amino acid sequence denoted by SEQ ID NO. 1, the method comprising the step of administering a therapeutically effective amount of at least one inhibitor of a Bcl2 prosurvival protein or any functional derivative thereof, or pharmaceutically acceptable salt thereof, or any compositions, kits or combinations thereof to said subject.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a graph showing the synergism between the drugs termed Nerofe and ABT-737 in inducing apoptosis of U937 cells, measured via activation of Caspase 3.

FIG. 2 is a graph showing that Nerofe synergizes with ABT-737 to induce death of U937 cells, measured via the percentage of cell viability with Resazurin.

FIG. 3 is a graph showing that Nerofe does not synergize with Azacitidine to induce death of U937 cells, measured via the percentage of cell viability with Resazurin.

FIG. 4 is a graph showing that Nerofe synergizes with ABT-737 to decrease viability of CT26 cells, measured via the percentage of cell viability with Resazurin.

FIG. 5 is a picture of a Western blot assay staining for Cleaved Caspase 3 of CT26 cells treated with Nerofe in presence/absence of ABT-737.

### DETAILED DESCRIPTION OF THE INVENTION

Bcl2 prosurvival protein inhibitors, a new class of anticancer drugs were shown herein to act synergistically together with a peptide termed “Nerofe” in inducing apoptosis and cell death of malignant cells.

Therefore in a first aspect, the present disclosure provides a combined composition comprising at least one inhibitor of a B-cell lymphoma 2 (Bcl2) prosurvival protein and an isolated peptide comprising the amino acid sequence denoted by SEQ ID NO. 1 or any functional derivative thereof, or pharmaceutically acceptable salt thereof and at least one pharmaceutically acceptable carrier, diluent, excipient and/or additive.

The term “isolated peptide” as herein defined encompasses an isolated peptide comprising the amino acid sequence denoted by SEQ ID NO. 1 (namely the amino acid sequence Trp Trp Thr Phe Phe Leu Pro Ser Thr Leu Trp Glu Arg Lys in an all D conformation), termed herein “dTCApFs” or “Nerofe” and functional derivatives of the amino acid sequence denoted by SEQ ID NO. 1 or pharmaceutically acceptable salts of said isolated peptide. Any pharmaceutically acceptable salt of the isolated peptide as herein defined are encompassed by the present disclosure, in particular the acetate salt of the peptide.

The term “peptide” as herein defined refers to a molecular chain of amino acid residues, which, if required, can be modified at each one of its amino acid residues, for example by manosylation, glycosylation, amidation (for example C-terminal amides), carboxylation or phosphorylation. The peptide may be obtained synthetically, through genetic engineering methods, expression in a host cell, or through any other suitable means. Methods for producing peptides are well known in the art.

More specifically, “Amino acid molecule”, “Amino acid sequence” or “peptide sequence” is the order in which amino acid residues connected by peptide bonds, lie in the chain in peptides and proteins. The sequence is generally reported from the N-terminal end containing free amino group to the C-terminal end containing amide. Amino acid sequence is often called peptide, protein sequence if it represents the primary structure of a protein, however one must discern between the terms “Amino acid sequence” or “peptide sequence” and “protein”, since a protein is defined as an amino acid sequence folded into a specific three-dimensional configuration and that had typically undergone post-translational modifications, such as phosphorylation, acetylation, glycosylation, manosylation, amidation, carboxylation, sulfhydryl bond formation, cleavage and the like.

Amino acids, as used herein refer to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. “Amino acid analogs” refers to compounds that have the same fundamental chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission.

The term “isolated” refers to molecules, such as amino acid sequences or peptides that are removed from their natural environment, isolated or separated.

The term “amino acid” as used herein, refers to naturally occurring and synthetic amino acid residues, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, Y-carboxyglutamate, and O-phosphoserine.

The term amino acid encompasses L-amino acids and D-amino acids, which are mirror images of L-amino acids, where the chirality at carbon alpha has been inverted. D-amino acids are highly resistant to protease mediated degradation and have a low immunogenic response.

The terms “amino acid sequence” or “peptide sequence” also relate to the order in which amino acid residues, connected by peptide bonds, lie in the chain in peptides and proteins. The sequence is generally reported from the N-terminal end containing free amino group to the C-terminal end containing free carboxyl group.

By the term “comprising” it is meant that the isolated peptide in accordance with the present disclosure includes the peptide denoted by SEQ ID NO: 1, but may also include additional amino acid residues at the N-terminus or at the C-terminus of the peptide or at both termini.

As indicated above, the present disclosure also encompasses isolated peptides comprising derivatives of the peptide having the amino acid sequence denoted by SEQ ID NO. 1. By the term “derivative” or “derivatives” it is meant to include peptides, which comprise the amino acid sequence denoted by SEQ ID NO: 1, but differ in one or more amino acids in their overall sequence, namely, which have deletions, substitutions (e.g. replacement of at least one amino acid by another amino acid), inversions or additions within the overall sequence of SEQ ID NO: 1. This term also encompasses the replacement of at least one amino acid residue in the overall sequence by its respective L amino acid residue.

In particular embodiments, the modified isolated peptide of the composition of the present disclosure or invention has at least 70%, or 80%, or 90%, or 95%, or in particular 99% identity to the corresponding sequence of SEQ ID NO: 1.

In some embodiments, the peptide comprising the amino acid sequence denoted by SEQ ID NO. 1 may comprise one or more amino acid residues is replaced by conservative substitution without significantly affecting the biological characteristics of the modified peptide as compared to the unmodified peptide having the amino acid sequence of SEQ ID NO: 1.

Amino acid “substitutions” are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, each of the following eight groups contains amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M).

It is appreciated that these peptide derivatives must not alter the biological activity of the original peptide. The term “functional” means to denote that the modified peptide (namely the derivative) retains a biological activity qualitatively similar to that of the unmodified peptide. The biological activity of the derivative may be determined as herein described, namely by monitoring the effect of said derivative upon administration to an animal model, as known in the art.

In particular embodiments, the present disclosure relates to a functional derivative thereof or functional fragment of the isolated polypeptide comprising amino acid sequence denoted by SEQ ID NO. 1, wherein said functional derivative thereof or functional fragment has an amino acid sequence that is at least about 70%, 75%, 80%, 85%, 90%, or 95%, in particular 99% identical to the amino acid sequence of the unmodified isolated polypeptide of the invention, namely to the amino acid sequence denoted by SEQ ID NO: 1 and retains a biological activity qualitatively similar to that of the unmodified peptide.

In some specific embodiments, the isolated peptide consists of the amino acid sequence of SEQ ID NO: 1.

Still further, as used herein the term Bcl-2 prosurvival protein refers to a proto-oncogenic protein known as an apoptosis inhibitor. The Bcl-2 protein forms the basis of a growing family of related proteins collectively denoted herein as Bcl-2 family of proteins. These proteins are known to control apoptotic cell death by the mitochondrial pathway.

As appreciated in the art, the members of the Bcl-2 family are either pro-survival or pro-apoptotic but regardless of their activity, they all share significant sequence and structural homology. Specifically, the Bcl-2 family of proteins is characterized by up to four regions of sequence homology, known as the Bcl-2 homology (BH) domains.

The Bcl-2 family of proteins includes three different groups of proteins: the first group is a pro-survival or anti-apoptotic group denoted herein as “Bcl-2 pro-survival proteins”, the second group is a pro-apoptotic group including BAX and BAK; and a third group denoted herein as BH3-only proteins that exhibit a pro-apoptotic activity.

The “Bcl-2 pro-survival proteins” or “anti-apoptotic” or “Bcl-2 like” as used herein denotes a group of proteins responsible for protecting cells from apoptotic stimuli and are sequentially characterized by containing all four BH domains.

It was previously shown that in response to an apoptotic stimulus, the balance of prosurvival and pro-apoptotic Bcl-2 proteins, and the specific interactions between them, determines the activity of the protein family. For example, as opposed to the Bcl-2 prosurvival proteins, the BH3-only proteins initiate apoptosis in response to diverse cellular stresses including DNA damage, growth factor deprivation, and endoplasmic reticulum stress. Activation of the BH3-only proteins may involve transcriptional up regulation or modification.

Several mechanisms have been suggested in the art to account for the diverse activity and tight control of apoptosis in the Bcl-2 family, some of which involve the Bax, Bak. Irrespective of the exact means it has become clear that the balance of prosurvival and pro-apoptotic (BH3-only) proteins is the key to this activation.

According to some embodiment, the Bcl2 inhibitor may antagonize any Bcl-2 prosurvival protein, for example, at least one of Bcl-2, Bcl-xL, Mcl-1, Bcl-w, Bcl2A1 and Bcl-B/Bcl2L10.

In some further embodiments, the Bcl-2 prosurvival protein of the composition of the invention is at least one of Bcl-2, B-cell lymphoma-extra large (Bcl-xL), Myeloid Cell Leukemia-1 (Mcl-1), B-cell lymphoma-w (Bcl-w), B-cell lymphoma 2 related protein A1 (Bcl2A1) and B-cell lymphoma 2 like 10 (Bcl-B/Bcl2L10).

More specifically, in some embodiments the Bcl2 inhibitor may bind and antagonize the Bcl-2 protein. Bcl-2 (B-cell CLL/lymphoma 2) as used herein, is an integral outer mitochondrial membrane protein that blocks the apoptotic death of some cells such as lymphocytes. Bcl-2 suppresses apoptosis in a variety of cell systems including factor-dependent lympho-hematopoietic and neural cells. It regulates cell death by controlling the mitochondrial membrane permeability. Bcl-2 appears to function in a feedback loop system with caspases, it inhibits caspase activity either by preventing the release of cytochrome c from the mitochondria and/or by binding to the apoptosis-activating factor (APAF-1). It should be noted that in certain embodiments, the invention refers to the human Bcl-2 protein as denoted by GenBank Accession No. NP_000624 and SEQ ID NO: 2 and NP_000648 of SEQ ID NO: 3, encoded by the Bcl-2 gene of GenBank Accession No. NM_000633 of SEQ ID NO: 4 and NM_000657 of SEQ ID NO: 5.

In yet another embodiment, the Bcl2 inhibitor may bind and antagonize Bcl-xL. B-cell lymphoma-extra large (Bcl-xL) as used herein, is a transmembrane molecule in the mitochondria. It is a member of the Bcl-2 family of proteins, and acts as a pro-survival protein by preventing the release of mitochondrial contents such as cytochrome c, which would lead to caspase activation. In certain embodiments the invention relates to the human Bcl-xL protein (GenBank Accession No. CAA80661 SEQ ID NO: 6), encoded by the Bcl-xL gene as denoted by GenBank Accession No. Z23115 and SEQ ID NO: 7.

In yet another embodiment, the Bcl2 inhibitor may bind and antagonize any one of the human Bcl-2 pro-survival proteins, Mcl-1, Bcl-w, Bcl2A1 and Bcl-B/Bcl2L10 as denoted by accession number: NP_068779.1, AAB09055, NP_004040.1 and NP_001293097.1, respectively.

In some other embodiments, the at least one inhibitor of a Bcl2 prosurvival protein is at least one Bcl-2 homology 3 (BH3) mimetic compound.

As mentioned above, BH3-mimetics are a class of anticancer drug that mimic the actions of BH3-only proteins in that they bind to prosurvival proteins like BCL2 in the same way and inhibit BCL2's ability to bind BAX or BAK.

In some specific embodiments, the BH3 mimetic compound is at least one of 4-{4-[(4′-Chloro[1,1′-biphenyl]-2-yl)methyl]piperazin-1-yl}-N-(4-{[(2R)-4-(dimethylamino)-1-(phenylsulfanyl) butan-2-yl]ami-no}-3-nitrobenzene-1-sulfonyl)benzamide (ABT-737), 4-(4-{[2-(4-Chlorophenyl)-4,4-dimethyl-1-cyclohexen-1-yl]methyl}-1-piperazinyl)-N-({3-nitro-4-[(tetrahydro-2H-pyran-4-ylmethyl)-amino]phenyl}sulfonyl)-2-(1H-pyrrolo[2,3-b]-pyridin-5-yloxy)benzamide (ABT-199), 4-(4-{[2-(4-Chlorophenyl)-5,5-dimethylcyclohex-1-en-1-yl]methyl}piperazin-1-yl)-N-(4-{[(2R)-4-(morpholin-4-yl)-1-(phe-nylsulfanyl) butan-2-yl]amino}-3-(trifluoromethanesulfonyl)benzene-1-sulfonyl)-benzamide (ABT-263), 2-(2-((3,5-Dimethyl-1H-pyrrol-2-yl)methylene)-3-methoxy-2H-pyrrol-5-yl)-1H-indole (GX15-070), or 3-[1-(1-adamantylmethyl)-5-methylpyrazol-4-yl]-6-[8-(1,3-benzothiazol-2-ylcarbamoyl)-3,4-dihydro-1H-isoquinolin-2-yl]pyridine-2-carboxylic acid (A-1331852), or any combinations thereof.

In some further embodiments, the at least one inhibitor of a Bcl2 prosurvival protein or BH3 mimetic compound is 4-{4-[(4′-Chloro[1,1′-biphenyl]-2-yl)methyl]piperazin-1-yl}-N-(4-{[(2R)-4-(dimethylamino)-1-(phenylsulfanyl) butan-2-yl]ami-no}-3-nitrobenzene-1-sulfonyl)-benzamide (ABT-737), or any a pharmaceutically acceptable salt or hydrate thereof or any stereoisomer or salt thereof.

ABT-737 is a small molecule drug that inhibits Bcl-2 and Bcl-xL, two members of the Bcl-2 family of evolutionarily-conserved proteins that share Bcl-2 Homology (BH) domains. ABT-737 is not bioavailable after oral administration, leading to the development of navitoclax (ABT-263) as an orally-available derivative with similar activity on small cell lung cancer (SCLC) cell lines.

ABT-737 has the following chemical structure, as denoted by Formula I:

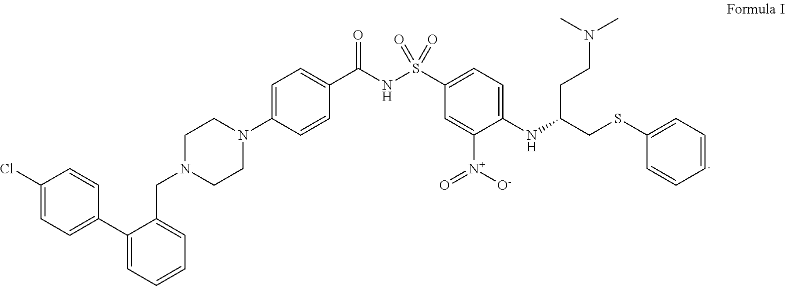 [SMILES: CN(C)CC[C@H](CSc1ccccc1)Nc1ccc(S(=O)(=O)NC(=O)c2ccc(N3CCN(Cc4ccccc4-c4ccc(Cl)cc4)CC3)cc2)cc1[N+](=O)[O-]]

The systematic (IUPAC) name of ABT-737 is 4-{4-[(4′-Chloro[1,1′-biphenyl]-2-yl)methyl]piperazin-1-yl}-N-(4-{[(2R)-4-(dimethylamino)-1-(phenylsulfanyl) butan-2-yl]ami-no}-3-nitrobenzene-1-sulfonyl)benzamide (C₄₂H₄₅ClN₆O₅S₂; CAS number: 852808 April 9). The molecular weight of ABT-737 is 813.43 g/mol.

In some further embodiments, the at least one inhibitor of a Bcl2 prosurvival protein or BH3 mimetic compound is 4-(4-{[2-(4-Chlorophenyl)-4,4-dimethyl-1-cyclohexen-1-yl]methyl}-1-piperazinyl)-N-({3-nitro-4-[(tetrahydro-2H-pyran-4-ylmethyl)-amino]phenyl}sulfonyl)-2-(1H-pyrrolo[2,3-b]-pyridin-5-yloxy)benzamide (ABT-199), or any a pharmaceutically acceptable salt or hydrate thereof or any stereoisomer or salt thereof.

ABT-199 or venetoclax, sold under the brand names Venclexta and Venclyxto, is a medication used to treat adults with chronic lymphocytic leukemia (CLL), small lymphocytic lymphoma (SLL), or acute myeloid leukemia (AML). Venetoclax attaches to the Bcl-2 protein which is present in high amounts in CLL cancer cells, where it helps the cells survive for longer in the body and makes them resistant to cancer medicines. By attaching to Bcl-2 and blocking its actions, venetoclax causes the death of cancer cells and thereby slows down progression of the disease. Venetoclax is the first selective BCL2 inhibitor, to be approved for routine clinical practice, specifically in chronic lymphocytic leukemia (CLL) and acute myeloid leukemia (AML).

ABT-199 has the following chemical structure, as denoted by Formula II:

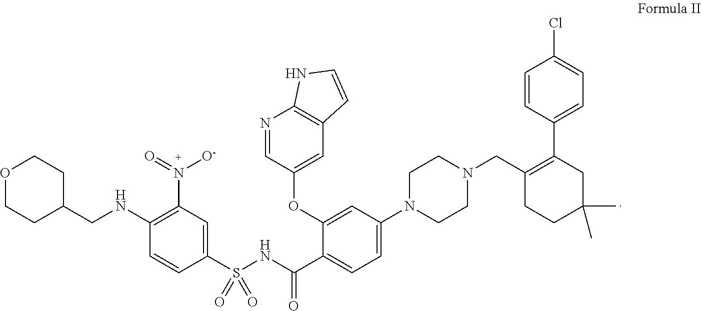

The systematic (IUPAC) name of ABT-199 is 4-(4-{[2-(4-Chlorophenyl)-4,4-dimethyl-1-cyclohexen-1-yl]methyl}-1-piperazinyl)-N-({3-nitro-4-[(tetrahydro-2H-pyran-4-ylmethyl)-amino]phenyl}sulfonyl)-2-(1H-pyrrolo[2,3-b]pyridin-5-yloxy)benzamide (C45H50ClN7O7S; CAS number: 1257044-40-8). The molecular weight of ABT-199 is 868.45 g/mol.

In some further embodiments, the at least one inhibitor of a Bcl2 prosurvival protein or BH3 mimetic compound is 4-(4-{[2-(4-Chlorophenyl)-5,5-dimethylcyclohex-1-en-1-yl]methyl}piperazin-1-yl)-N-(4-{[(2R)-4-(morpholin-4-yl)-1-(phe-nylsulfanyl) butan-2-yl]amino}-3-(trifluoromethanesulfonyl)benzene-1-sulfonyl)-benzamide (ABT-263), or any a pharmaceutically acceptable salt or hydrate thereof or any stereoisomer or salt thereof.

ABT-263 or navitoclax is an orally active anti-cancer drug, which is a Bcl-2 inhibitor. ABT-263 has the following chemical structure, as denoted by Formula III:

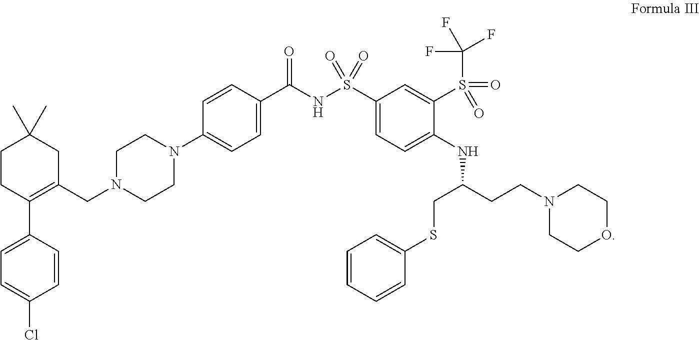 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

The systematic (IUPAC) name of ABT-263 is 4-(4-{[2-(4-Chlorophenyl)-5,5-dimethylcyclohex-1-en-1-yl]methyl}piperazin-1-yl)-N-(4-{[(2R)-4-(morpholin-4-yl)-1-(phe-nylsulfanyl) butan-2-yl]amino}-3-(trifluoromethanesulfonyl)benzene-1-sulfonyl)benzamide (C₄₇H₅₅ClF₃N₅O₆S₃; CAS number: 923564-51-6). The molecular weight of ABT-263 is 974.61 g/mol.

In some further embodiments, the at least one inhibitor of a Bcl2 prosurvival protein or BH3 mimetic compound is 2-(2-((3,5-Dimethyl-1H-pyrrol-2-yl)methylene)-3-methoxy-2H-pyrrol-5-yl)-1H-indole (GX15-070), or any a pharmaceutically acceptable salt or hydrate thereof or any stereoisomer or salt thereof.

GX15-070 or obatoclax is a drug for the treatment of various types of cancer. Obatoclax is an inhibitor of the Bcl-2 family of proteins. This inhibition induces apoptosis in cancer cells, preventing tumor growth. GX15-070 has the following chemical structure, as denoted by Formula IV:

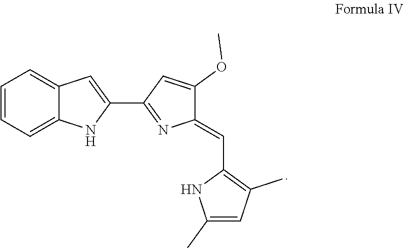 [SMILES: COC1=CC(c2cc3ccccc3[nH]2)=N/C1=C\c1[nH]c(C)cc1C]

The systematic (IUPAC) name of GX15-070 is 2-(2-((3,5-Dimethyl-1H-pyrrol-2-yl)methylene)-3-methoxy-2H-pyrrol-5-yl)-1H-indole (C₂₀H₁₉N₃O; CAS number: 803712 July 6). The molecular weight of GX15-070 is 317.392 g/mol.

In some further embodiments, the at least one inhibitor of a Bcl2 prosurvival protein or BH3 mimetic compound is 3-[1-(1-adamantylmethyl)-5-methylpyrazol-4-yl]-6-[8-(1,3-benzothiazol-2-ylcarbamoyl)-3,4-dihydro-1H-isoquinolin-2-yl]pyridine-2-carboxylic acid (A-1331852), or any a pharmaceutically acceptable salt or hydrate thereof or any stereoisomer or salt thereof.

A-1331852 is a first-in-class orally active BCL-XL inhibitor that selectively and potently induces apoptosis in BCL-XL-dependent tumor cells. A-1331852 has the following chemical structure, as denoted by Formula V:

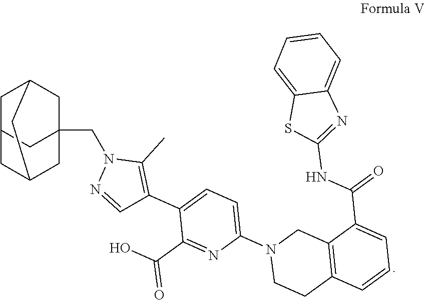 [SMILES: Cc1c(-c2ccc(N3CCc4cccc(C(=O)Nc5nc6ccccc6s5)c4C3)nc2C(=O)O)cnn1CC12CC3CC(CC(C3)C1)C2]

The systematic (IUPAC) name of A-1331852 is 3-[1-(1-adamantylmethyl)-5-methylpyrazol-4-yl]-6-[8-(1,3-benzothiazol-2-ylcarbamoyl)-3,4-dihydro-1H-isoquino-lin-2-yl]pyridine-2-carboxylic acid (C₃₈H₃₈N₆O₃S; CAS number: 1430844-80-6). The molecular weight of A-1331852 is 658.27 g/mol.

In some embodiments, the composition may comprise at least one additional therapeutic agents e.g. anti-cancer agents. In some further embodiments, said composition does not comprise taxol, also known as Paclitaxel, or doxorubicin or an anti PDL1 antibody.

In yet some further embodiments, the composition of the present disclosure is for use in a method of treating, preventing, ameliorating, reducing or delaying the onset of at least one proliferative disorder.

In some embodiments, the proliferative disorder relevant to the composition of the invention is associated with Bcl2 over-expression.

In some further embodiments, the proliferative disorder is at least one hematological cancer or neoplasm.

In some specific embodiments, the hematological cancer or neoplasm is at least one of chronic lymphocytic leukemia (CLL), acute myeloid leukemia (AML), mutated KRAS tumor, lymphomas, multiple myeloma, blastic plasmacytoid dendritic cell neoplasm, T-cell prolymphocytic leukemia or myelodysplasia.

As shown in the examples below, the combined composition was shown to act synergistically to induce apoptosis and cell death of U937 and CT26 cells which are cell lines derived from histiocytic lymphoma and colorectal carcinoma respectively.

In some specific embodiments, the proliferative disorder is at least one histiocytic lymphoma or colorectal carcinoma.

It should be noted that the combined composition of the invention may induce or enhance apoptosis of malignant/cancer cells. More specifically, the combined composition of the invention may lead to an increase, enhancement, induction or elevation in apoptosis of treated cells, said increase, induction or elevation of apoptosis may be an increase by about 1% to 99.9%, specifically, about 1% to about 95%, about 5% to 90%, about 10% to 85%, about 15% to 80%, about 20% to 75%, about 25% to 70%, about 30% to 65%, about 35% to 60%, about 40% to 55%, about 45% to 50%. More specifically, about 5%, 10%, 15%, 20%, 25%, 30%, 35%, 40%, 45%, 50%, 55%, 60%, 65%, 70%, 75%, 80%, 85%, 90%, 95% or 99.9%. More specifically, an increase of about 5%, 10%, 15%, 20%, 25%, 30%, 35%, 40%, 45%, 50%, 55%, 60%, 65%, 70%, 75%, 80%, 85%, 90%, 95% or 99% as compared to untreated control.

With regards to the above, it is to be understood that, where provided, percentage values such as, for example, 10%, 50%, 120%, 500%, etc., are interchangeable with “fold change” values, i.e., 0.1, 0.5, 1.2, 5, 10, 20, 30, 40, 50, 60, 70, 80, 90, 100 etc., respectively.

The term “apoptosis” refers to a regulated network of biochemical events which lead to a selective form of cell suicide and is characterized by readily observable morphological and biochemical phenomena. Cells undergoing apoptosis show characteristic morphological and biochemical features. These features include chromatin aggregation or condensation, DNA fragmentation, nuclear and cytoplasmic condensation, partition of cytoplasm and nucleus into membrane bound vesicles (apoptotic bodies) which contain ribosomes, morphologically intact mitochondria and nuclear material. Cytochrome C release from mitochondria is seen as an indication of mitochondrial dysfunction accompanying apoptosis.

As indicated above, apoptosis is a tightly controlled form of active cell death that is necessary for development and organismal homeostasis. Death by the apoptotic pathway is achieved among others, by the activation of a family of highly potent and specific proteases, termed caspases (for cysteine-aspartate protease).

The activity of caspases is tightly regulated and the cell maintains several “checkpoints” to control their activity. The first level of regulation is intrinsic to caspases themselves. Caspases are initially transcribed as weakly active zymogens, which only upon proper stimulation are cleaved to form the active enzyme. The second level of caspase regulation is achieved by inhibitors, namely by a family of proteins called IAPs (Inhibitor of Apoptosis Protein).

Therefore in a second aspect, the present disclosure provides a method of treating, preventing, ameliorating, reducing or delaying the onset of at least one proliferative disorder in a subject in need thereof, comprising the step of administering a therapeutically effective amount of at least one inhibitor of a Bcl2 prosurvival protein and an isolated peptide comprising the amino acid sequence denoted by SEQ ID NO. 1 or any functional derivative thereof, or pharmaceutically acceptable salt thereof, or any compositions, kits or combinations thereof.

In some embodiments, the modified isolated peptide suitable for the method of the present disclosure or invention has at least 70%, or 80%, or 90%, or 95% identity to the corresponding sequence of SEQ ID NO: 1.

In some specific embodiments, the isolated peptide consists of the amino acid sequence of SEQ ID NO: 1.

In another embodiments, the Bcl-2 prosurvival protein relevant to the method of the invention is at least one of Bcl-2, Bcl-XL, Mcl-1, Bcl-w, Bcl2A1 and Bcl-B/Bcl2L10.

In some further embodiments, the at least one inhibitor of a Bcl2 prosurvival protein suitable for the method of the invention is at least one BH3 mimetic compound.

In some further specific embodiments, the BH3 mimetic compound is at least one of 4-{4-[(4′-Chloro[1,1′-biphenyl]-2-yl)methyl]piperazin-1-yl}-N-(4-{[(2R)-4-(dimethylamino)-1-(phenylsulfanyl) butan-2-yl]ami-no}-3-nitrobenzene-1-sulfonyl)benzamide (ABT-737), 4-(4-{[2-(4-Chlorophenyl)-4,4-dimethyl-1-cyclohexen-1-yl]methyl}-1-piperazinyl)-N-({3-nitro-4-[(tetrahydro-2H-pyran-4-ylmethyl)-amino]phenyl}sulfonyl)-2-(1H-pyrrolo[2,3-b]-pyridin-5-yloxy)benzamide (ABT-199), 4-(4-{[2-(4-Chlorophenyl)-5,5-dimethylcyclohex-1-en-1-yl]methyl}piperazin-1-yl)-N-(4-{[(2R)-4-(morpholin-4-yl)-1-(phe-nylsulfanyl) butan-2-yl]amino}-3-(trifluoromethanesulfonyl)benzene-1-sulfonyl)-benzamide (ABT-263), 2-(2-((3,5-Dimethyl-1H-pyrrol-2-yl)methylene)-3-methoxy-2H-pyrrol-5-yl)-1H-indole (GX15-070), or 3-[1-(1-adamantylmethyl)-5-methylpyrazol-4-yl]-6-[8-(1,3-benzothiazol-2-ylcarbamoyl)-3,4-dihydro-1H-isoquinolin-2-yl]pyridine-2-carboxylic acid (A-1331852).

In yet another specific embodiment, the at least one inhibitor of a Bcl2 prosurvival protein is 4-{4-[(4′-Chloro[1,1′-biphenyl]-2-yl)methyl]piperazin-1-yl}-N-(4-{[(2R)-4-(dimethylamino)-1-(phenylsulfanyl) butan-2-yl]ami-no}-3-nitrobenzene-1-sulfonyl)-benzamide (ABT-737), or any a pharmaceutically acceptable salt or hydrate thereof or any stereoisomer or salt thereof.

As used herein, “proliferative disorder” is a disorder displaying hyper proliferation. This term means cell division and growth that is not part of normal cellular turnover, metabolism, growth, or propagation of the whole organism. Unwanted proliferation of cells is seen in tumors and other pathological proliferation of cells, does not serve normal function, and for the most part will continue unbridled at a growth rate exceeding that of cells of a normal tissue in the absence of outside intervention. A pathological state that ensues because of the unwanted proliferation of cells is referred herein as a “hyper proliferative disease” or “hyper proliferative disorder.” It should be noted that the term “proliferative disorder”, “cancer”, “tumor” and “malignancy” all relate equivalently to a hyperplasia of a tissue or organ. In general, the compositions and methods of the present invention may be used in the treatment of non-solid and solid tumors.

Malignancy, as contemplated in the present invention may be any one of lymphomas, leukemias, carcinomas, melanomas, myeloma and sarcomas.

Lymphoma is a cancer in the lymphatic cells of the immune system. Typically, lymphomas present as a solid tumor of lymphoid cells. These malignant cells often originate in lymph nodes, presenting as an enlargement of the node (a tumor). It can also affect other organs in which case it is referred to as extranodal lymphoma. Non limiting examples for lymphoma include Hodgkin's disease, non-Hodgkin's lymphomas and Burkitt's lymphoma.

Leukemia refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number of abnormal cells in the blood-leukemic or aleukemic (subleukemic).

Carcinoma as used herein refers to an invasive malignant tumor consisting of transformed epithelial cells. Alternatively, it refers to a malignant tumor composed of transformed cells of unknown histogenesis, but which possess specific molecular or histological characteristics that are associated with epithelial cells, such as the production of cytokeratins or intercellular bridges.

Melanoma as used herein is a malignant tumor of melanocytes. Melanocytes are cells that produce the dark pigment, melanin, which is responsible for the color of skin. They predominantly occur in skin, but are also found in other parts of the body, including the bowel and the eye. Melanoma can occur in any part of the body that contains melanocytes.

Sarcoma is a cancer that arises from transformed connective tissue cells. These cells originate from embryonic mesoderm, or middle layer, which forms the bone, cartilage, and fat tissues. This is in contrast to carcinomas, which originate in the epithelium. The epithelium lines the surface of structures throughout the body, and is the origin of cancers in the breast, colon, and pancreas.

Myeloma as mentioned herein is a cancer of plasma cells, a type of white blood cell normally responsible for the production of antibodies. Collections of abnormal cells accumulate in bones, where they cause bone lesions, and in the bone marrow where they interfere with the production of normal blood cells. Most cases of myeloma also feature the production of a paraprotein, an abnormal antibody that can cause kidney problems and interferes with the production of normal antibodies leading to immunodeficiency. Hypercalcemia (high calcium levels) is often encountered.

Further malignancies that may find utility in the present invention can comprise but are not limited to hematological malignancies (including lymphoma, leukemia and myeloproliferative disorders), hypoplastic and aplastic anemia (both virally induced and idiopathic), myelodysplastic syndromes, all types of paraneoplastic syndromes (both immune mediated and idiopathic) and solid tumors (including GI tract, colon, lung, liver, breast, prostate, pancreas and Kaposi's sarcoma. More particularly, the malignant disorder may be lymphoma. Non-limiting examples of cancers treatable according to the invention include hematopoietic malignancies such as all types of lymphomas, leukemia, e.g. acute lymphocytic leukemia (ALL), acute myelogenous leukemia (AML), chronic lymphocytic leukemia (CLL), mutated KRAS tumors, chronic myelogenous leukemia (CML), myelodysplastic syndrome (MDS), mast cell leukemia, hairy cell leukemia, Hodgkin's disease, non-Hodgkin's lymphomas, Burkitt's lymphoma and multiple myeloma, as well as for the treatment or inhibition of solid tumors such as tumors in lip and oral cavity, pharynx, larynx, paranasal sinuses, major salivary glands, thyroid gland, esophagus, stomach, small intestine, colon, colorectum, anal canal, liver, gallbladder, extraliepatic bile ducts, ampulla of vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma and Kaposi's sarcoma.

Specifically, it was shown that Bcl-2 proteins that are anti-apoptotic proteins govern the pro-survival pathway and are over expressed in a variety of tumor types such small cell lung cancer, melanoma, prostate and breast cancer.

In some embodiments, the proliferative disorder relevant to the method of the invention is associated with Bcl2 over-expression or Bcl-2-over-expressing-disorder or Bcl-2-mediated disorder.

The terms “disorders associated with Bcl2 over-expression” or “Bcl-2-over-expressing-disorder” and “Bcl-2-mediated disorder” refer to pathological and disease conditions in which a Bcl-2 protein is over-expressed as indicated herein above. Moreover, this term also encompasses conditions in which Bcl-2 plays a role. Such roles can be directly related to the pathological condition or can be indirectly related to the condition. The feature common to this class of conditions is that they can be ameliorated by inhibiting the expression of, activity of, function of, or association with Bcl-2 proteins.

The term “over expressed” refers to an increase in the measurable expression level of Bcl-2 gene as measured by the amount of RNA and/or the amount of protein in a sample as compared with the measurable expression level of Bcl-2 gene in a second sample, specifically, a control sample. “Over expressed Bcl-2” can be measured and evaluated using the ratio of the level of expression of Bcl-2 in a sample as compared with the mean expression level of Bcl-2 of a control sample wherein the ratio is not equal and specifically, is above 1.0. When determining over expression on the basis of the ratio, an RNA or protein is over expressed if the ratio of the level of expression in a first sample as compared with a second sample is greater than 1.0. For example, a ratio of greater than 1.2, 1.5, 1.7, 2, 3, 4, 5, 6, 7, 8, 9, 10, 20, 30, 40, 50, 60, 70, 80, 90, 100 or more.

Thus, a Bcl-2 over-expressing pathological disorder is meant a disorder characterized by over-expression of Bcl-2 in said subject or in a diseased tissue of said subject as compared to a healthy subject or a healthy tissue of the same subject.

It should be noted that the Bcl-2 over-expressing disorder may be caused by chromosomal translocation, hypo-methylation and down regulation of the microRNAs that target Bcl-2.

B-cell lymphoma 2 (BCL2) proteins are known to be variably highly expressed in many hematological malignancies, providing protection from cell death induced by oncogenic and external stresses. For example, Venetoclax is the first selective BCL2 inhibitor, and the first of the class of anticancer drug (BH3-mimetics) that was approved for routine clinical practice, in chronic lymphocytic leukemia (CLL) and acute myeloid leukemia (AML).

In some particular embodiments, the compositions or methods of the invention are for treating, preventing, ameliorating, reducing or delaying the onset of chronic lymphocytic leukemia (CLL). Chronic lymphocytic leukemia (CLL) is a type of cancer in which the bone marrow produce an excessive amount of lymphocytes.

In some particular embodiments, the compositions or methods of the invention are for treating, preventing, ameliorating, reducing or delaying the onset of acute myeloid leukemia (AML). Acute myeloid leukemia (AML) is a cancer of the myeloid line of blood cells, characterized by the rapid growth of abnormal cells that build up in the bone marrow and blood and interfere with normal blood cell production.

In some particular embodiments, the compositions or methods of the invention are for treating, preventing, ameliorating, reducing or delaying the onset of mutated KRAS tumors. Mutated KRAS tumors or KRAS-driven tumors relate to a type of cancer tumors in which the Kirsten rat sarcoma viral oncogene (KRAS) is mutated. The KRAS oncogene has the highest mutation rate among all cancers and is associated with a series of highly fatal cancers, such as pancreatic ductal adenocarcinoma (PDAC), nonsmall-cell lung cancer (NSCLC), and colorectal cancer (CRC).

Thus, in some specific embodiments, the proliferative disorder is at least one hematological cancer or neoplasm.

In yet some specific embodiments, the hematological cancer or neoplasm is at least one of chronic lymphocytic leukemia (CLL), acute myeloid leukemia (AML), mutated KRAS tumors, lymphomas, multiple myeloma, blastic plasmacytoid dendritic cell neoplasm, T-cell prolymphocytic leukemia or myelodysplasia.

In some particular embodiments, the proliferative disorder relevant to the method of the invention is histiocytic lymphoma or colorectal carcinoma.

In some further embodiments, the method of the invention enhances or induces apoptosis of malignant/cancer cells of said subject.

In yet another embodiment, the method of the invention further comprises administering at least one therapeutic agent e.g. at least one additional anti-cancer agent.

In some specific embodiments, said additional anticancer agent is not taxol or doxorubicin or an anti PDL1 antibody.

In some embodiments of the method of the invention, the at least one inhibitor of a Bcl2 prosurvival protein and an isolated peptide comprising the amino acid sequence denoted by SEQ ID NO. 1 are administered concomitantly or consecutively.

In a third aspect, the present disclosure provides at least one inhibitor of a Bcl2 prosurvival protein and at least one isolated peptide comprising the amino acid sequence denoted by SEQ ID NO. 1 or any functional derivative thereof for use in a method of treating, preventing, ameliorating, reducing or delaying the onset of at least one proliferative disorder, or a pharmaceutically acceptable salt thereof.

In some embodiments, the modified isolated peptide suitable for the use according to the present disclosure or invention has at least 70%, or 80%, or 90%, or 95% identity to the corresponding sequence of SEQ ID NO: 1.

In some specific embodiments, the isolated peptide consists of the amino acid sequence of SEQ ID NO: 1.

In another embodiments, the Bcl-2 prosurvival protein relevant to the use according to the invention is at least one of Bcl-2, Bcl-xL, Mcl-1, Bcl-w, Bcl2A1 and Bcl-B/Bcl2L10.

In some further embodiments, the at least one inhibitor of a Bcl2 prosurvival protein suitable for the use according to the invention is at least one BH3 mimetic compound.

In some further specific embodiments, the BH3 mimetic compound is at least one of 4-{4-[(4′-Chloro[1,1′-biphenyl]-2-yl)methyl]piperazin-1-yl}-N-(4-{[(2R)-4-(dimethylamino)-1-(phenylsulfanyl) butan-2-yl]ami-no}-3-nitrobenzene-1-sulfonyl)benzamide (ABT-737), 4-(4-{[2-(4-Chlorophenyl)-4,4-dimethyl-1-cyclohexen-1-yl]methyl}-1-piperazinyl)-N-({3-nitro-4-[(tetrahydro-2H-pyran-4-ylmethyl)-amino]phenyl}sulfonyl)-2-(1H-pyrrolo[2,3-b]-pyridin-5-yloxy)benzamide (ABT-199), 4-(4-{[2-(4-Chlorophenyl)-5,5-dimethylcyclohex-1-en-1-yl]methyl}piperazin-1-yl)-N-(4-{[(2R)-4-(morpholin-4-yl)-1-(phe-nylsulfanyl) butan-2-yl]amino}-3-(trifluoromethanesulfonyl)benzene-1-sulfonyl)-benzamide (ABT-263), 2-(2-((3,5-Dimethyl-1H-pyrrol-2-yl)methylene)-3-methoxy-2H-pyrrol-5-yl)-1H-indole (GX15-070), or 3-[1-(1-adamantylmethyl)-5-methylpyrazol-4-yl]-6-[8-(1,3-benzothiazol-2-ylcarbamoyl)-3,4-dihydro-1H-isoquinolin-2-yl]pyridine-2-carboxylic acid (A-1331852).

In yet another specific embodiment, the at least one inhibitor of a Bcl2 prosurvival protein is 4-{4-[(4′-Chloro[1,1′-biphenyl]-2-yl)methyl]piperazin-1-yl}-N-(4-{[(2R)-4-(dimethylamino)-1-(phenylsulfanyl) butan-2-yl]ami-no}-3-nitrobenzene-1-sulfonyl)-benzamide (ABT-737), or any a pharmaceutically acceptable salt or hydrate thereof or any stereoisomer or salt thereof.

In some embodiments, the at least one inhibitor of a Bcl2 prosurvival protein and an isolated peptide comprising the amino acid sequence denoted by SEQ ID NO. 1 for use according to the invention are administered concomitantly or consecutively.

In yet another embodiment, the use of the at least one inhibitor of a Bcl2 prosurvival protein and an isolated peptide comprising the amino acid sequence denoted by SEQ ID NO. 1 according to the invention further comprises administering at least one additional anti-cancer agent.

In some specific embodiments, said additional anticancer agent is not taxol or doxorubicin or an anti PDL1 antibody.

In some embodiments, the proliferative disorder relevant to the use according to the invention is associated with Bcl2 over-expression.

In some specific embodiments, the proliferative disorder is at least one hematological cancer or neoplasm.

In yet some specific embodiments, the hematological cancer or neoplasm is at least one of chronic lymphocytic leukemia (CLL), acute myeloid leukemia (AML), mutated KRAS tumors, lymphomas, multiple myeloma, blastic plasmacytoid dendritic cell neoplasm, T-cell prolymphocytic leukemia or myelodysplasia.

In some particular embodiments, the proliferative suitable for the use of the invention is histiocytic lymphoma and colorectal carcinoma respectively.

In some embodiments the isolated peptide or Bcl2 inhibitor or a pharmaceutically acceptable salt thereof as herein defined and/or suitable for the methods and uses disclosed herein may be comprised in a pharmaceutical composition. The term “pharmaceutical compositions” as herein defined refers to the isolated peptide and/or the Bcl2 inhibitor and optionally at least one pharmaceutically acceptable excipient or carrier as known in the art. As used herein “pharmaceutically acceptable carrier” includes any and all solvents, dispersion media, coatings, antibacterial and antifungal agents and the like. The use of such media and agents for pharmaceutical active substances is well known in the art. Except as any conventional media or agent is incompatible with the active ingredient, its use in the therapeutic composition is contemplated. Pharmaceutical compositions used to treat subjects in need thereof according to the present disclosure optionally also comprise a buffering agent, an agent who adjusts the osmolarity thereof, and optionally, one or more pharmaceutically acceptable additives as known in the art. Pharmaceutical compositions used to treat subjects in need thereof according to the invention, which may conveniently be presented in unit dosage form, may be prepared according to conventional techniques well known in the pharmaceutical industry, for example as detailed in the Examples below. It should be understood that in addition to the ingredients particularly mentioned herein, the compositions according to the present disclosure may also include other agents conventional in the art having regard to the type of formulation in question.

The isolated peptide comprising the amino acid sequence denoted by SEQ ID NO. 1 and/or the Bcl2 inhibitor defined herein may be administered by any route of administration known to a person skilled in the art, for example intravenously (iv) or any suitable additional route including intraperitoneal, subcutaneous, transcutaneous, topical, intramuscular, intraarticular, subconjunctival, or mucosal, e.g. oral, intranasal, or intraocular administration.

The isolated peptide or Bcl2 inhibitor as herein defined may be administered at an “effective amount” such that necessary to achieve the desired therapeutic result. The “effective amount” is determined by the severity of the disease in conjunction with the therapeutic objectives, the route of administration and the patient's general condition (age, sex, weight and other considerations known to the attending physician). Amounts effective for this use generally range from 0.001 to 1000 mg/Kg, specifically from 0.001 mg/Kg to 0.1 mg/kg, 0.02 mg/Kg to 0.1 mg/kg, 0.03 mg/Kg to 0.1 mg/kg, 0.04 mg/Kg to 0.1 mg/kg, 0.05 mg/Kg to 0.1 mg/kg, 0.06 mg/Kg to 0.1 mg/kg, 0.07 mg/Kg to 0.1 mg/kg, 0.08 mg/Kg, 0.09 mg/Kg to 0.1 mg/kg, from 0.1 mg/Kg to 1 mg/kg, 0.2 mg/Kg to 1 mg/kg, 0.3 mg/Kg to 1 mg/kg, 0.4 mg/Kg to 1 mg/kg, 0.5 mg/Kg to 1 mg/kg, 0.6 mg/Kg to 1 mg/kg, 0.7 mg/Kg to 1 mg/kg, 0.8 mg/Kg to 1 mg/kg, 0.9 mg/Kg to 1 mg/kg, 1 mg/Kg to 10 mg/kg, 2 mg/Kg to 10 mg/kg, 3 mg/Kg to 10 mg/kg, 4 mg/Kg to 10 mg/kg, 5 mg/Kg to 10 mg/kg, 6 mg/Kg to 10 mg/kg, 7 mg/Kg to 10 mg/kg, 8 mg/Kg to 10 mg/kg, 9 mg/Kg to 10 mg/kg, 10 mg/Kg to 100 mg/kg, 20 mg/Kg to 100 mg/kg, 30 mg/Kg to 100 mg/kg, 40 mg/Kg to 100 mg/kg, 50 mg/Kg to 100 mg/kg, 60 mg/Kg to 100 mg/kg, 70 mg/Kg to 100 mg/kg, 80 mg/Kg to 100 mg/kg, 90 mg/Kg to 100 mg/Kg, or from 100 mg/Kg to 1000 mg/Kg, 200 mg/Kg to 1000 mg/Kg, 300 mg/Kg to 1000 mg/Kg, 400 mg/Kg to 1000 mg/Kg, 500 mg/Kg to 1000 mg/Kg, 600 mg/Kg to 1000 mg/Kg, 700 mg/Kg to 1000 mg/Kg, 800 mg/Kg to 1000 mg/Kg, 900 mg/Kg to 1000 mg/Kg. In some specific embodiment, the amount or dose of is 1 mg/Kg, 2 mg/Kg, 3 mg/Kg, 4 mg/Kg, 5 mg/Kg, 6 mg/Kg, 7 mg/Kg, 8 mg/Kg, 9 mg/Kg, 10 mg/Kg, 15 mg/kg, 20 mg/Kg, 30 mg/Kg, 40 mg/Kg, 50 mg/Kg, 60 mg/Kg, 70 mg/Kg, 80 mg/Kg, 90 mg/Kg, or 100 mg/Kg. In some particular embodiments, the amount or dose of Nerofe is 15 mg/kg. In some further embodiments, the amount of Bcl2 inhibitor is 50 mg/kg.

In some further embodiment, an effective amount refers to an amount of Nerofe and Bcl2 inhibitor for obtaining a synergistic effect.

Single or multiple administrations on a daily, weekly or monthly schedule can be carried out with dose levels and pattern being selected by the treating physician. Additionally, the administration of the compositions of the invention, may be periodic, for example, the periodic administration may be effected twice daily, three time daily, or at least one daily for at least about three days to three months. The advantages of lower doses are evident to those of skill in the art. These include, inter alia, a lower risk of side effects, especially in long-term use, and a lower risk of the patients becoming desensitized to the treatment. In another embodiment, treatment using the compositions of the invention, may be effected following at least 1, 2, 3, 4, 5, 6, 7, 8, 9, 10, 14, 30, 60, 90 days of treatment, and proceeding on to treatment for life. It should be noted that the treatment may indicate the use of different doses or different time periods, these will be evident to the skilled medical practitioner.

For prophylactic applications, the compositions of the invention may include a prophylactic effective amount of the active ingredient. The term “prophylactically effective amount” is intended to mean that amount of a pharmaceutical composition that will prevent or reduce the risk of occurrence or recurrence of the biological or medical event that is sought to be prevented in a tissue, a system, animal or human by a researcher, veterinarian, medical doctor or other clinician. In prophylactic applications, the compositions of the invention are administered to a patient who is at risk of developing the disease state to enhance the patient's resistance. Such an amount is defined to be a “prophylactically effective dose”. In this use, the precise amounts again depend upon the patient's state of health and general level of immunity, but generally range from 0.001 to 1000 mg per dose.

The phrase “combination therapy” or “adjunct therapy” or in defining use of a peptide described herein, specifically, the isolated peptide comprising the amino acid sequence as denoted by SEQ ID NO: 1 also termed “Nerofe” and at least one Bcl2 inhibitor specifically inhibitors of Bcl-2 pro-apoptotic proteins, is intended to embrace administration of each agent in a sequential manner in a regimen that will provide beneficial effects of the drug combination, and is intended as well to embrace co-administration of these agents in a substantially simultaneous manner, such as in a single formulation having a fixed ratio of these active agents, or in multiple, separate formulations for each agent.

Therefore in a further aspect, in a fifth aspect, the present disclosure provides a method of treating, preventing, ameliorating, reducing or delaying the onset of at least one proliferative disorder in a subject treated with at least one inhibitor of a Bcl2 prosurvival protein, the method comprising the step of administering a therapeutically effective amount of an isolated peptide comprising the amino acid sequence denoted by SEQ ID NO. 1 or any functional derivative thereof, or pharmaceutically acceptable salt thereof, or any compositions, kits or combinations thereof, to said subject.

In yet another aspect, the present disclosure provides a method of treating, preventing, ameliorating, reducing or delaying the onset of at least one proliferative disorder in a subject treated with at least one isolated peptide comprising the amino acid sequence denoted by SEQ ID NO. 1, the method comprising the step of administering a therapeutically effective amount of at least one inhibitor of a Bcl2 prosurvival protein or any functional derivative thereof, or pharmaceutically acceptable salt thereof, or any compositions, kits or combinations thereof to said subject.

With respect with the above aspects of the present disclosure relating to a method of treating, preventing, ameliorating, reducing or delaying the onset of at least one proliferative disorder in a subject treated with at least one inhibitor of a Bcl2 prosurvival protein or to a method of treating, preventing, ameliorating, reducing or delaying the onset of at least one proliferative disorder in a subject treated with at least one isolated peptide comprising the amino acid sequence denoted by SEQ ID NO. 1, in some embodiments said modified isolated peptide has at least 70%, or 80%, or 90%, or 95% identity to the corresponding sequence of SEQ ID NO: 1. In some embodiments, said isolated peptide consists of the amino acid sequence of SEQ ID NO: 1. In some embodiments, said Bcl-2 prosurvival protein is at least one of Bcl-2, Bcl-xL, Mcl-1, Bcl-w, Bcl2A1 and Bcl-B/Bcl2L 10. In some embodiments, said at least one inhibitor of a Bcl2 prosurvival protein is at least one BH3 mimetic compound. In some embodiments, said BH3 mimetic compound is at least one of 4-{4-[(4′-Chloro[1,1′-biphenyl]-2-yl)methyl]piperazin-1-yl}-N-(4-{[(2R)-4-(dimethylamino)-1-(phenylsulfanyl) butan-2-yl]ami-no}-3-nitrobenzene-1-sulfonyl)benzamide (ABT-737), 4-(4-{[2-(4-Chlorophenyl)-4,4-dimethyl-1-cyclohexen-1-yl]methyl}-1-piperazinyl)-N-({3-nitro-4-[(tetrahydro-2H-pyran-4-ylmethyl)-amino]phenyl}sulfonyl)-2-(1H-pyrrolo[2,3-b]-pyridin-5-yloxy)benzamide (ABT-199), 4-(4-{[2-(4-Chlorophenyl)-5,5-dimethylcyclohex-1-en-1-yl]methyl}piperazin-1-yl)-N-(4-{[(2R)-4-(morpholin-4-yl)-1-(phe-nylsulfanyl) butan-2-yl]amino}-3-(trifluoromethanesulfonyl)benzene-1-sulfonyl)-benzamide (ABT-263), 2-(2-((3,5-Dimethyl-1H-pyrrol-2-yl)methylene)-3-methoxy-2H-pyrrol-5-yl)-1H-indole (GX15-070), or 3-[1-(1-adamantylmethyl)-5-methylpyrazol-4-yl]-6-[8-(1,3-benzothiazol-2-ylcarbamoyl)-3,4-dihydro-1H-isoquinolin-2-yl]pyridine-2-carboxylic acid (A-1331852). In some embodiments, said at least one inhibitor of a Bcl2 prosurvival protein is 4-{4-[(4′-Chloro[1,1′-biphenyl]-2-yl)methyl]piperazin-1-yl}-N-(4-{[(2R)-4-(dimethylamino)-1-(phenylsulfanyl) butan-2-yl]ami-no}-3-nitrobenzene-1-sulfonyl)-benzamide (ABT-737), or any a pharmaceutically acceptable salt or hydrate thereof or any stereoisomer or salt thereof. In some embodiments, said at least one inhibitor of a Bel2 prosurvival protein and an isolated peptide comprising the amino acid sequence denoted by SEQ ID NO. 1 are administered concomitantly or consecutively. In some embodiments, the method further comprises administering at least one additional anti-cancer agent. In some embodiments, the proliferative disorder is associated with Bcl2 over-expression. In some embodiments, said proliferative disorder is at least one hematological cancer or neoplasm. In some embodiments, said hematological cancer or neoplasm is at least one of chronic lymphocytic leukemia (CLL), acute myeloid leukemia (AML), mutated KRAS tumors, lymphomas, multiple myeloma, blastic plasmacytoid dendritic cell neoplasm, T-cell prolymphocytic leukemia or myelodysplasia.

Therefore in a further aspect, the present disclosure provides an isolated peptide comprising the amino acid sequence denoted by SEQ ID NO. 1 for use in a method of treating, preventing, ameliorating, reducing or delaying the onset of at least one proliferative disorder in a subject treated with at least one inhibitor of a Bcl2 prosurvival protein, the method comprising the step of administering a therapeutically effective amount of said isolated peptide comprising the amino acid sequence denoted by SEQ ID NO. 1 or any functional derivative thereof, or pharmaceutically acceptable salt thereof, or any compositions, kits or combinations thereof, to said subject.

In yet another aspect, the present disclosure provides a Bcl2 prosurvival protein for use in a method of treating, preventing, ameliorating, reducing or delaying the onset of at least one proliferative disorder in a subject treated with at least one isolated peptide comprising the amino acid sequence denoted by SEQ ID NO. 1, the method comprising the step of administering a therapeutically effective amount of said at least one inhibitor of a Bel2 prosurvival protein or any functional derivative thereof, or pharmaceutically acceptable salt thereof, or any compositions, kits or combinations thereof to said subject.

In some embodiments, said modified isolated peptide suitable for the uses of the invention has at least 70%, or 80%, or 90%, or 95% identity to the corresponding sequence of SEQ ID NO: 1. In some embodiments, said Bcl-2 prosurvival protein suitable for the uses of the invention is at least one of Bcl-2, Bcl-xL, Mcl-1, Bcl-w, Bcl2A1 and Bcl-B/Bcl2L10. In some embodiments, said at least one inhibitor of a Bcl2 prosurvival protein suitable for the uses of the invention is at least one BH3 mimetic compound. In some embodiments, said BH3 mimetic compound suitable for the uses of the invention is at least one of 4-{4-[(4′-Chloro[1,1′-biphenyl]-2-yl)methyl]piperazin-1-yl}-N-(4-{[(2R)-4-(dimethylamino)-1-(phenylsulfanyl) butan-2-yl]ami-no}-3-nitrobenzene-1-sulfonyl)benzamide (ABT-737), 4-(4-{[2-(4-Chlorophenyl)-4,4-dimethyl-1-cyclohexen-1-yl]methyl}-1-piperazinyl)-N-({3-nitro-4-[(tetrahydro-2H-pyran-4-ylmethyl)-amino]phenyl}sulfonyl)-2-(1H-pyrrolo[2,3-b]-pyridin-5-yloxy)benzamide (ABT-199), 4-(4-{[2-(4-Chlorophenyl)-5,5-dimethylcyclohex-1-en-1-yl]methyl}piperazin-1-yl)-N-(4-{[(2R)-4-(morpholin-4-yl)-1-(phe-nylsulfanyl) butan-2-yl]amino}-3-(trifluoromethanesulfonyl)benzene-1-sulfonyl)-benzamide (ABT-263), 2-(2-((3,5-Dimethyl-1H-pyrrol-2-yl)methylene)-3-methoxy-2H-pyrrol-5-yl)-1H-indole (GX15-070), or 3-[1-(1-adamantylmethyl)-5-methylpyrazol-4-yl]-6-[8-(1,3-benzothiazol-2-ylcarbamoyl)-3,4-dihydro-1H-isoquinolin-2-yl]pyridine-2-carboxylic acid (A-1331852). In some embodiments, said at least one inhibitor of a Bcl2 prosurvival protein suitable for the uses of the invention is 4-{4-[(4′-Chloro[1,1′-biphenyl]-2-yl)methyl]piperazin-1-yl}-N-(4-{[(2R)-4-(dimethylamino)-1-(phenylsulfanyl) butan-2-yl]ami-no}-3-nitrobenzene-1-sulfonyl)-benzamide (ABT-737), or any a pharmaceutically acceptable salt or hydrate thereof or any stereoisomer or salt thereof. In some embodiments, said at least one inhibitor of a Bcl2 prosurvival protein and an isolated peptide suitable for the uses of the invention comprising the amino acid sequence denoted by SEQ ID NO. 1 are administered concomitantly or consecutively. In some embodiments, the uses in accordance with the present invention further comprises administering at least one additional anti-cancer agent. In some embodiments, proliferative disorder relevant to the uses of the invention is associated with Bcl2 over-expression. In some embodiments, said proliferative disorder relevant to the uses of the invention is at least one hematological cancer or neoplasm. In some embodiments, said hematological cancer or neoplasm relevant to the uses of the invention is at least one of chronic lymphocytic leukemia (CLL), acute myeloid leukemia (AML), mutated KRAS tumors, lymphomas, multiple myeloma, blastic plasmacytoid dendritic cell neoplasm, T-cell prolymphocytic leukemia or myelodysplasia.

As noted above, the present invention involves the use of different active ingredients, for example, the peptide termed “Nerofe” and at least one Bcl2 inhibitor that may be administered through different routes, dosages and combinations. More specifically, the treatment of Bcl-2 over-expressing diseases and conditions with a combination of active ingredients may involve separate administration of each active ingredient. Therefore, a kit providing a convenient modular format would allow the required flexibility in the above parameters.

More specifically, the kits further described below herein can include a composition as described, or in separate multiple dosage unit forms, as an already prepared liquid topical, nasal or oral dosage form ready for administration or, alternatively, can include the composition as described as a solid pharmaceutical composition that can be reconstituted with a solvent to provide a liquid dosage form. When the kit includes a solid pharmaceutical composition that can be reconstituted with a solvent to provide a liquid dosage form (e.g., for oral administration), the kit may optionally include a reconstituting solvent. In this case, the constituting or reconstituting solvent is combined with the active ingredient to provide liquid dosage forms of each of the active ingredients or of a combination thereof. Typically, the active ingredients are soluble in so the solvent and forms a solution. The solvent can be, e.g., water, a non-aqueous liquid, or a combination of a non-aqueous component and an aqueous component. Suitable non-aqueous components include, but are not limited to oils, alcohols, such as ethanol, glycerin, and glycols, such as polyethylene glycol and propylene glycol. In some embodiments, the solvent is phosphate buffered saline (PBS).

In a further aspect, the present disclosure provides a kit comprising:
(a) at least one inhibitor of a Bcl2 prosurvival protein or any pharmaceutically acceptable salt thereof, optionally, in a first dosage form;
(b) an isolated peptide comprising the amino acid sequence denoted by SEQ ID NO. 1 or any functional derivative thereof, or pharmaceutically acceptable salt thereof, optionally, in a second dosage form.

In some embodiments, the modified isolated peptide suitable for the kit of the present disclosure or invention has at least 70%, or 80%, or 90%, or 95% identity to the corresponding sequence of SEQ ID NO: 1.

In some specific embodiments, the isolated peptide consists of the amino acid sequence of SEQ ID NO: 1.

In some further embodiments, the said Bcl-2 prosurvival protein relevant to the kits of the invention is at least one of Bcl-2, Bcl-XL, Mcl-1, Bcl-w, Bcl2A1 and Bcl-B/Bcl2L10.

In some yet specific embodiment, the at least one inhibitor of a Bcl2 prosurvival protein is at least one BH3 mimetic compound.

In some more specific embodiments, the BH3 mimetic compound suitable for the kit of the invention is at least one of 4-{4-[(4′-Chloro[1,1′-biphenyl]-2-yl)methyl]piperazin-1-yl}-N-(4-{[(2R)-4-(dimethylamino)-1-(phenylsulfanyl) butan-2-yl]ami-no}-3-nitrobenzene-1-sulfonyl)benzamide (ABT-737), 4-(4-{[2-(4-Chlorophenyl)-4,4-dimethyl-1-cyclohexen-1-yl]methyl}-1-piperazinyl)-N-({3-nitro-4-[(tetrahydro-2H-pyran-4-ylmethyl)-amino]phenyl}sulfonyl)-2-(1H-pyrrolo[2,3-b]-pyridin-5-yloxy)benzamide (ABT-199), 4-(4-{[2-(4-Chlorophenyl)-5,5-dimethylcyclohex-1-en-1-yl]methyl}piperazin-1-yl)-N-(4-{[(2R)-4-(morpholin-4-yl)-1-(phe-nylsulfanyl) butan-2-yl]amino}-3-(trifluoromethanesulfonyl)benzene-1-sulfonyl)-benzamide (ABT-263), 2-(2-((3,5-Dimethyl-1H-pyrrol-2-yl)methylene)-3-methoxy-2H-pyrrol-5-yl)-1H-indole (GX15-070), or 3-[1-(1-adamantylmethyl)-5-methylpyrazol-4-yl]-6-[8-(1,3-benzothiazol-2-ylcarbamoyl)-3,4-dihydro-1H-isoquinolin-2-yl]pyridine-2-carboxylic acid (A-1331852).

In some particular embodiments, the at least one inhibitor of a Bcl2 prosurvival protein is 4-{4-[(4′-Chloro[1,1′-biphenyl]-2-yl)methyl]piperazin-1-yl}-N-(4-{[(2R)-4-(dimethylamino)-1-(phenylsulfanyl) butan-2-yl]ami-no}-3-nitrobenzene-1-sulfonyl)-benzamide (ABT-737), or any a pharmaceutically acceptable salt or hydrate thereof or any stereoisomer or salt thereof.

In some further embodiments, the kit according to the present disclosure is for use in a method of treating, preventing, ameliorating, reducing or delaying the onset of at least one proliferative disorder.

In some embodiments, the at least one inhibitor of a Bcl2 prosurvival protein and said isolated peptide comprising the amino acid sequence denoted by SEQ ID NO. 1 are in separated unit dosage forms in the kit for use according to the invention.

In some further embodiments, the separated unit dosage forms of at least one inhibitor of a Bcl2 prosurvival protein and an isolated peptide comprising the amino acid sequence denoted by SEQ ID NO. 1 are administered concomitantly or consecutively.

In some other embodiments, the proliferative disorder relevant to the kit for use of the invention is associated with Bcl2 over-expression.

In some further embodiments, the proliferative disorder is at least one hematological cancer or neoplasm.

In some specific embodiments, the hematological cancer or neoplasm is at least one of chronic lymphocytic leukemia (CLL), acute myeloid leukemia (AML), mutated KRAS tumors, lymphomas, multiple myeloma, blastic plasmacytoid dendritic cell neoplasm, T-cell prolymphocytic leukemia or myelodysplasia.

The term “treatment or prevention” refers to the complete range of therapeutically positive effects of administrating to a subject including inhibition, reduction of, alleviation of, and relief from, proliferative disorder symptoms or undesired side effects of such proliferative disorder related disorders. More specifically, treatment or prevention includes the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms.

As used herein, “disease”, “disorder”, “condition” and the like, as they relate to a subject's health, are used interchangeably and have meanings ascribed to each and all of such terms.

The present invention relates to the treatment of subjects, or patients, in need thereof. By “patient” or “subject in need” it is meant any organism who may be affected by the above-mentioned conditions, and to whom the treatment methods herein described are desired, including humans, domestic and non-domestic mammals such as canine and feline subjects, bovine, simian, equine and murine subjects, rodents, domestic birds, aquaculture, fish and exotic aquarium fish. It should be appreciated that the treated subject may be also any reptile or zoo animal. More specifically, the methods and compositions of the invention are intended for mammals. By “mammalian subject” is meant any mammal for which the proposed therapy is desired, including human, equine, canine, and feline subjects, most specifically humans. It should be noted that specifically in cases of non-human subjects, the method of the invention may be performed using administration via injection, drinking water, feed, spraying, oral gavage and directly into the digestive tract of subjects in need thereof. It should be further noted that particularly in case of human subject, administering of the compositions of the invention to the patient includes both self-administration and administration to the patient by another person.

All definitions, as defined and used herein, should be understood to control over dictionary definitions, definitions in documents incorporated by reference, and/or ordinary meanings of the defined terms.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. In some embodiments, the term “about” refers to +10%.

The indefinite articles “a” and “an,” as used herein in the specification and in the claims, unless clearly indicated to the contrary, should be understood to mean “at least one.” It must be noted that, as used in this specification and the appended claims, the singular forms “a”, “an” and “the” include plural referents unless the content clearly dictates otherwise.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

Throughout this specification and the Examples and claims which follow, all transitional phrases such as “comprising,” “including,” “carrying,” “having,” “containing,” “involving,” “holding,” “composed of,” and the like are to be understood to be open-ended, i.e., to mean including but not limited to. Specifically, it should understood to imply the inclusion of a stated integer or step or group of integers or steps but not the exclusion of any other integer or step or group of integers or steps. Only the transitional phrases “consisting of” and “consisting essentially of” shall be closed or semi-closed transitional phrases, respectively, as set forth in the United States Patent Office Manual of Patent Examining Procedures. More specifically, the terms “comprises”, “comprising”, “includes”, “including”, “having” and their conjugates mean “including but not limited to”. The term “consisting of means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated herein above and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

### Experimental Procedures.

Preparation of the dTCApFs Peptide and Composition Comprising Thereof

The peptide dTCApFs or NEROFE™ (both terms are used herein interchangeably and refer to the same peptide as indicated above) is a 14 amino acid residues long peptide, in which all of the amino acid residues are at their D configuration, having the amino acid sequence of Trp Trp Thr Phe Phe Leu Pro Ser Thr Leu Trp Glu Arg Lys (or WWTFFLPSTLWERK in a single letter code, as denoted by SEQ ID NO: 1).

The peptide was synthesized as follows. dTCApFs Acetate, the final drug product for use in clinical studies, was manufactured, packaged, tested, labeled and released under Good Manufacturing Practices (GMP) by Nextar Ltd., Israel.

Cleaved Caspase 3 Western blot of U937 cells treated with Nerofe and ABT-737

### Materials:

For cell Growth:
Growth Medium—
   RPMI Media 1640 (Gibco 21875-034)
   50 ml heat inactivated FBS (Biological industries cat no. 04-121-1A).
   0.5 ml Amphotericin B 2500 μg/ml (Biological industries cat no. 03-029-1).
   Penicillin-Streptomycin Solution (Biological Industries cat no. 03-031)
Treatment Medium—
   Growth Medium
   10% Mannitol (Sigma cat no. M4125-500G)
      Filter the media in 0.2 μm filter after adding Mannitol.
175 cm² culture Flasks
20 mg/ml Nerofe in Mannitol
ABT-737 (Cayman 11501)
U937 cells line

### For Western Blot

Lysis Buffer containing:
   Ripa Buffer (Sigma R0278)
   Protease Inhibitor Cocktail (Sigma P2714).
   phosphatase inhibitors (Sigma P5726)
   Benzonase® Nuclease, Purity >90% (70746 Millipore) 25U/ml
X4 LDS sample buffer (invitrogen cat #NP0007)
Sample reducing agent (Invitrogen cat #NP0009, 10-fold concentrated)
4-12% Bis-Tris gel (Millipore cat #MMMP41G10)
10×MES transfer BUFFER (Millipore MMMPTRB)
PVDF membrane (immobilon-P sq 0.2 μm)
TBST buffer: TBS (J640 Amersco)+0.05% Tween-20
5% skim milk in TBST
1st antibodies diluted 1:500 in 5% skim milk in TBST:
   Anti-cleaved caspase 3 antibody (ab32042 abcam)
Secondary antibodies diluted 1:10,000 in 5% skim milk in TBST:
   Anti-rabbit IgG, HRP-linked Antibody (Cells signaling 7074)
SuperSignal™ West Femto Maximum Sensitivity Substrate (Thermo Scientific 34095)

### Cell Culture—Passages:

The cells were grown cells at 37° C., 5% CO₂ 100% humidity until 70-80% density is reached. Cells were resuspended with fresh medium and then left for growth 2-3 days until 70-80% is reached. The passage procedure was repeated.

### Cells Treatment:

The cells were seeded as 700K cells/75 CM2 flask in 13 ml growing media in 4 different flasks and incubated overnight. Mannitol was added sp the final mannitol concentration was of 5% Mannitol. The cells were then treated as follow:
1—Control (untreated)
2—50 μg/ml Nerofe
3—No treatment (for ABT-737 the following day)
4—50 g/ml Nerofe (for ABT-737 the following day)

The cells were then incubated for 24 hours and treated as follow:
1—Control (untreated)
2—Do not treat (treated previous day with Nerofe)
3—1 μM ABT-737
4—1 μM ABT-737 (treated previous day with Nerofe)

The cells were then incubated for 24 hours and collected for performing Western Blot as previously described (Mahmood, T., & Yang, P. C. (2012). Western blot: technique, theory, and trouble shooting. North American journal of medical sciences, 4 (9), 429-434).

Cell Viability Assay (Resazurin) of U937 Cells Treated with Nerofe and Azacytidine

### Materials:

Cell Culture Media:
10% FBS (Biological industries, cat #04-121-1A)
DMEM (Gibco cat #41965)
1 mM sodium pyruvate (Biological Industries)
100 μg/ml penicillin and 100 μg/ml streptomycin (Biological Industries)
250 ng/ml Amphotericin B (Biological Industries)
Treatment Medium—
Culture media
10% Mannitol (Sigma cat no. M4125-500G)

Filter the media in 0.2 μm filter after adding Mannitol.
Cell Cytotoxicity Assay Kit (Colorimetric): ab112118 abcam
Cell line: U937
2 mg/ml Nerofe stock solution: Dilute 100 ul of 20 mg/ml Nerofe in 900 μl Cell treatment media
5-Azacytidine (Sigma A2385, freshly diluted to 50 mg/mlm in treatment media)

Treatment regimen:

| μg/ml Nerofe 48 hrs | nM Azacytidine 48 hrs |
|---|---|
| 0 | 0 |
| 50 | 0 |
| 0 | 50 |
| 0 | 100 |
| 50 | 50 |
| 50 | 100 |
| No cells | |

The cells were seeded at 20000 cells/well in a black 96 well plate with clear bottom in 100 μl growing media and incubated for 24 hours. The cells were treated with Nerofe and Azacytidin in 100 μl cell treatment media with 10% mannitol at a final volume of 200 μl and final mannitol concentration 5%. The cells were incubated 48 hours. An amount of 20 μl of Cell Cytotoxicity Assay Kit (Colorimetric) ab112118 was added to each well, and the cells were incubated for 4 hours. The plates were read at excitation 530 emission 590, sensitivity 40 using Biotek Synergy-HT plate reader. The percentage of cells viability was calculated compared to control.
Cell Viability assay (Resazurin) of U937 cells treated with Nerofe and ABT-737 Materials

The materials were the same as above except for ABT-737 (Cayman 11501) instead of 5-Azacytidine

Treatment regimen:

| μg/ml Nerofe 48 hrs | μM ABT-737 24 hrs |
|---|---|
| 0 | 0 |
| 50 | 0 |
| 0 | 0.5 |
| 0 | 1 |
| 0 | 2 |
| 50 | 0.5 |
| 50 | 1 |
| 50 | 2 |
| No cells | |

The cells were seeded at 20000 cells/well in a black 96 well plate with clear bottom in 100 μl growing media and incubated for 24 hours. The cells were treated with Nerofe in 100 μl cell treatment media with 10% mannitol at a final volume of 200 μl and final mannitol concentration 5%. The cells were incubated 24 hours. The cells were treated with ABT-737 by adding 25 μl of treatment media with 5% mannitol without FBS to each well accordingly at a final volume of 225 μl. The cells were incubated for 24 hours. An amount of 25 μl of Cell Cytotoxicity Assay Kit (Colorimetric) ab112118 was added to each well, and the cells were incubated for 4 hours. The plates were read at excitation 530 emission 590, sensitivity 40 using Biotek Synergy-HT plate reader. The percentage of cells viability was calculated compared to control.

Cleaved Caspase 3 Western Blot of CT26 Cells Treated with Nerofe and ABT-737

### Materials:

For cell Growth:
Growth Medium—
   RPMI Media 1640 (Gibco 21875-034)
   50 ml heat inactivated FBS (Biological industries cat no. 04-121-1A).
   0.5 ml Amphotericin B 2500 μg/ml (Biological industries cat no. 03-029-1).
   Penicillin-Streptomycin Solution (Biological Industries cat no. 03-031)
Treatment Medium—
   Growth Medium
   5% Mannitol (Sigma cat no. M4125-500G)
Filter the media in 0.2 μm filter after adding Mannitol.
Trypsin EDTA (Biological industries cat no. 03-052-1B).
175 cm² culture Flasks
20 mg/ml Nerofe in Mannitol (use aliquots, avoid repeated freeze-thaw cycle).
ABT-737 (Cayman 11501)
CT26 cells line

### For Western Blot

Lysis Buffer containing:
   Ripa Buffer (Sigma R0278)
   Protease Inhibitor Cocktail (Sigma P2714).
   phosphatase inhibitors (Sigma P5726)
   Benzonase® Nuclease, Purity >90% (70746 Millipore) 25U/ml
X4 LDS sample buffer (invitrogen cat #NP0007)
Sample reducing agent (Invitrogen cat #NP0009, 10-fold concentrated)
4-12% Bis-Tris gel (Millipore cat #MMMP41G10)
10×MES transfer BUFFER (Millipore MMMPTRB)
PVDF membrane (immobilon-P sq 0.2 um)
TBST buffer: TBS (J640 Amersco)+0.05% Tween-20
5% skim milk in TBST
1st antibodies diluted 1:1000 in 5% skim milk in TBST:
   Anti-cleaved caspase 3 antibody (asp175) Cell Signaling #9661
Secondary antibodies diluted 1:10,000 in 5% skim milk in TBST:
   Anti-rabbit IgG, HRP-linked Antibody (Cells signaling 7074)
SuperSignal™ West Femto Maximum Sensitivity Substrate (Thermo Scientific 34095)

### Cell Culture-Passages:

The cells were grown cells at 37° C., 5% CO₂ 100% humidity until 70-80% density is reached. Cells were treated with Trypsin. Cells were resuspended with fresh medium and then left for growth 2-3 days until 70-80% is reached. The passage procedure was repeated.

### Cells Treatment:

The cells were seeded as 1M cells/75 CM₂ flask in 25 ml growing media in 4 different flasks and incubated overnight. Media was changed. The cells were then treated as follow:
1—Control (untreated)
2—50 μg/ml Nerofe
3—Do not treat (for ABT)
4—50 μg/ml Nerofe (for ABT)
5—Do not treat (for ABT)
6—50 μg/ml Nerofe (for ABT)

The cells were then incubated for 24 hours and treated as follow:
1—Control (untreated)
2—Do not treat
3—Do not treat
4—Do not treat
5—1 μM ABT
6—1 μM ABT

The cells were then incubated for 24 hours and treated as follow:
1—Control (untreated)
2—50 μg/ml Nerofe
3—1 μM ABT
4—1 μM ABT+50 μg/ml Nerofe
5—Do not treat
6—50 μg/ml Nerofe

The cells were then collected for performing Western Blot as previously described (Mahmood, T., & Yang, P. C. (2012). Western blot: technique, theory, and trouble shooting. North American journal of medical sciences, 4 (9), 429-434).

Cell Viability Assay (Resazurin) of CT26 Cells Treated with Nerofe and ABT-737

### Materials

Cell Culture Media:
10% FBS (Biological industries, cat #04-121-1A)
DMEM (Gibco cat #41965)
1 mM sodium pyruvate (Biological Industries)
100 μg/ml penicillin and 100 μg/ml streptomycin (Biological Industries)
250 ng/ml Amphotericin B (Biological Industries)
Treatment Medium—
5% Mannitol (Sigma cat no. M4125-500G) in culture media with FBS or
5% Mannitol (Sigma cat no. M4125-500G) in culture media without FBS

Filter the media in 0.2 μm filter after adding Mannitol.
Cell Cytotoxicity Assay Kit (Colorimetric): ab112118 abcam
Cell line: CT26
2 mg/ml Nerofe stock solution: Dilute 100 ul of 20 mg/ml Nerofe in 900 μl Cell treatment media
ABT-737 (Cayman 11501)

Treatment regimen:

| μg/ml Nerofe 24 + 72 hrs | μM ABT-737 24 hrs |
|---|---|
| 0 | 0 |
| 50 | 0 |
| 0 | 1 |
| 50 | 1 |
| No cells | |
| μg/ml Nerofe 24 + 72 hrs | μM ABT-737 48 hrs |
| 0 | 0 |
| 50 | 0 |
| 0 | 1 |
| 50 | 1 |
| No cells | |

The cells were seeded at 5000 cells/well in a black 96 well plate with clear bottom in 100 μl growing media. The cells were incubated for 24 hours. Media was removed and the cells were treated with Nerofe in 100 μl cell treatment media with FBS. The cells were incubated for 24 hours. The cells were then treated with ABT-737 in the 48 hours treatment wells by adding 25 μl of treatment media with 5% mannitol without FBS to each well accordingly at a final volume of 125 μl. The cells were incubated for 24 hours. The cells were treated with ABT-737 in the 24 hours treatment wells and with Nerofe by adding 25 μl of treatment media with 5% mannitol without FBS to each well accordingly at a final volume of 150 μl. The cells were incubated for 24 hours.

An amount of 25 μl of Cell Cytotoxicity Assay Kit (Colorimetric) ab112118 was added to each well, and the cells were incubated for 4 hours. The plates were read at excitation 530 emission 590, sensitivity 40 using Biotek Synergy-HT plate reader. The percentage of cells viability was calculated compared to control.

### Example 1

### Synergism Between Nerofe and a BCL2 Inhibitor in Inducing Apoptosis of U937 Cells

As indicated above, a peptide termed “T101” that is encoded by a cDNA unique to the human thymus was identified and was reported, inter alia, to reduce cancer tumor size. In addition, a peptide derivative of T101, termed herein dTCApFs (or “Nerofe”), has been reported to migration of cancer cells [8] and to be implicated in the induction of ER stress which contributes to promoting cell death [9].

Specifically, Nerofe is a peptide has the all D amino acid sequence of Trp Trp Thr Phe Phe Leu Pro Ser Thr Leu Trp Glu Arg Lys, as denoted by SEQ ID NO: 1 and was prepared as described above.

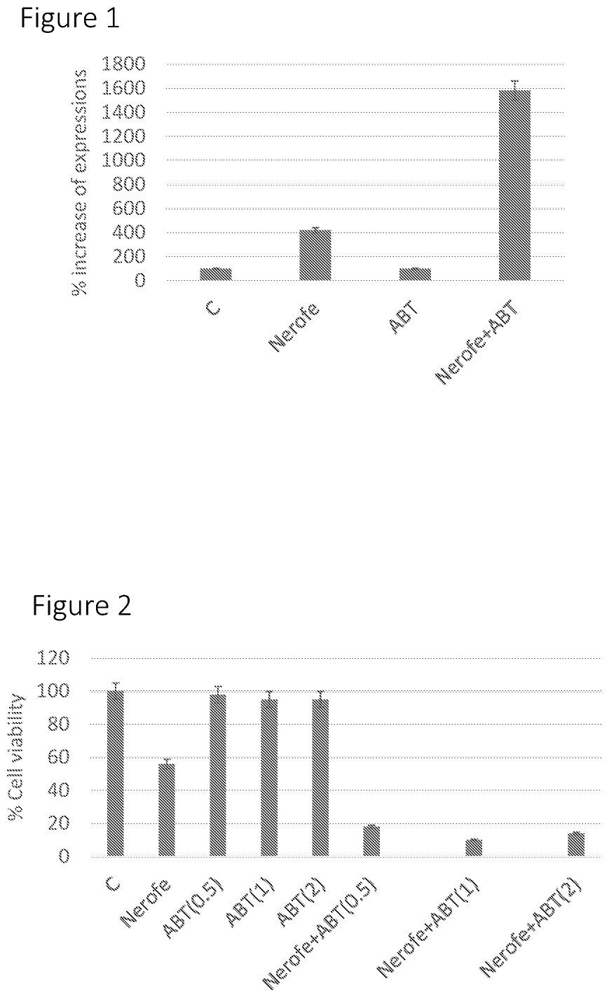

The effect of Nerofe and ABT-737 either alone or in combination on activation of Caspase 3 in U937 cells was examined. As shown in FIG. 1, a strong synergistic effect was observed when Nerofe was combined with a BCL2 inhibitor i.e. ABT-737 on the activation of Caspase which is a marker of apoptosis. Without wishing to be bound by theory, it is believed that BCL2 proteins are the main inhibitor of the apoptotic effect of Nerofe.

### Example 2

### Synergism Between Nerofe and a BCL2 Inhibitor in Inducing Cell Death of U937 Cells

The effect of Nerofe and a BCL2 inhibitor i.e. ABT-737 either alone or in combination on percentage of cell viability of U937 cells was examined at different concentration of ABT-737 i.e. 0.5, 1, or 2 μM. As shown in FIG. 2, a strong synergistic effect was observed when Nerofe was combined with ABT-737 on the induction of cell death.

### Example 3

### No Synergism is Observed Between Nerofe and Azacitidine in Inducing Cell Death

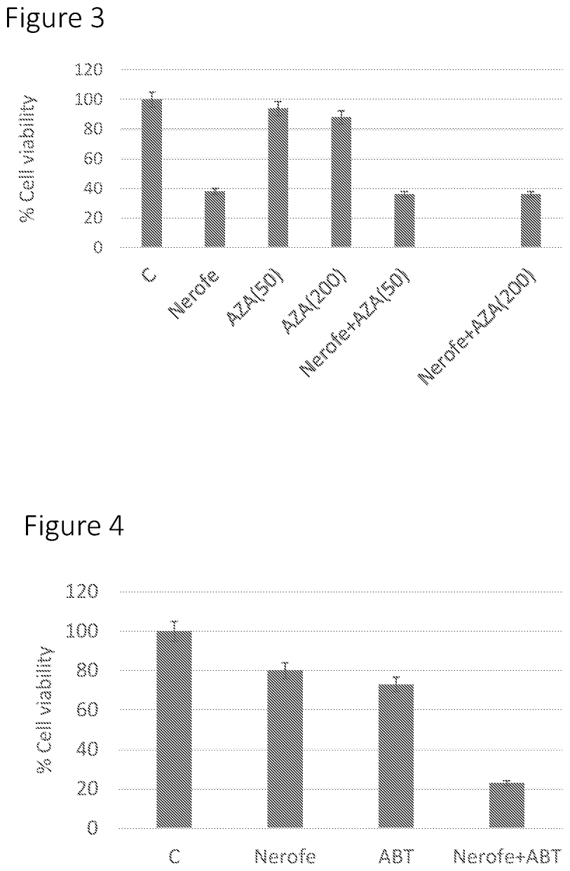

The effect of Nerofe and Azacitidine either alone or in combination on percentage of cell viability of U937 cells was examined at different concentration of Azacytidine i.e. 50 or 100 nM. As shown in FIG. 3, no synergistic effect was observed when Nerofe was combined with Azacytidine on the induction of cell death. Without wishing to be bound by theory, it seems that the antineoplastic activity of Nerofe is not via the same mechanism of action as Azacytidine. Azacytidine is a chemical analogue of the nucleoside cytidine, which is present in DNA and RNA which has antineoplastic activity via two mechanisms—at low doses, by inhibiting of DNA methyltransferase, causing hypomethylation of DNA, and at high doses, by its direct cytotoxicity through its incorporation into DNA and RNA, resulting in cell death. On the other end, Nerofe is believed to act via production of free oxygen radicals that destroy the Golgi apparatus and induces un-repaired ER stress in cancer cells over expressing T1/ST2 receptor.

### Example 4

### Synergism Between Nerofe and a BCL2 Inhibitor in Inducing Cell Death of CT26 Cells

The effect of Nerofe and a BCL2 inhibitor i.e. ABT-737 either alone or in combination on percentage of cell viability of CT26 cells was examined at a concentration of ABT-737 of 1 μM. As shown in FIG. 4, a strong synergistic effect was observed when Nerofe was combined with ABT-737 on the induction of cell death.

### Example 5

### Synergism Between Nerofe and a BCL2 Inhibitor in Inducing Apoptosis of CT26 Cells

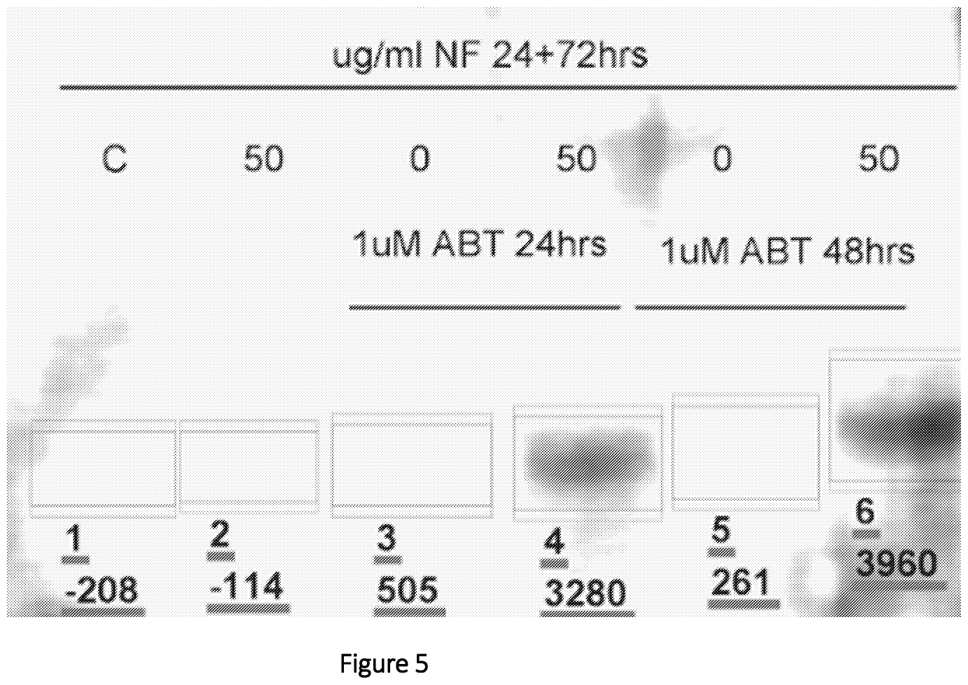

The effect of Nerofe and ABT-737 either alone or in combination on activation of Cleaved Caspase 3 in CT26 cells was examined by Western blot analysis. As shown in FIG. 5, a strong synergistic effect was observed when Nerofe was combined with a BCL2 inhibitor i.e. ABT-737 on the activation of the Caspase.

### Example 6

Effect of Nerofe with Additional Bcl2 Inhibitors

The effect of Nerofe with additional Bcl2 inhibitors such as ABT-199 (venetoclax), ABT-263 (navitoclax), GX15-070 (obatoclax) or A-1331852 in inducing apoptosis and/or cell death is further examined for example as described above.

### Example 7

### Effect of a Combination of Nerofe and a BCL2 Inhibitor in a Mouse Model

A number of 32 Blab/C females are inoculated subcutaneously (SC) with 100,000 CT26 cells. Once tumors have achieved volume of 50 mm³, the mice are divided into the following groups:
8 mice-Control
8 mice-Nerofe (15 mg/kg intraperitoneally (IP) 3 times a week
8 mice-ABT-737 (50 mg/Kg) IP 3 times a week
8 mice-Nerofe (15 mg/kg IP 3 times a week+ABT-737 (50 mg/Kg) IP 3 times a week.

Weight of mice is measured twice a week and tumor volume is also measured twice a week.

### Example 8

### Effect of a Combination of Nerofe and Additional BCL2 Inhibitors in a Mouse Model

A number of 32 Blab/C females are inoculated subcutaneously (SC) with 100,000 CT26 cells. Once tumors have achieved volume of 50 mm³, the mice are divided into the following groups:
8 mice-Control
8 mice-Nerofe (15 mg/kg intraperitoneally (IP) 3 times a week
8 mice-ABT-199 (venetoclax) IP 3 times a week
8 mice-ABT-263 (navitoclax) IP 3 times a week IP 3 times a week
8 mice-GX15-070 (obatoclax) IP 3 times a week IP 3 times a week
8 mice-A-1331852 IP 3 times a week IP 3 times a week
8 mice-Nerofe IP 3 times a week+ABT-199 (venetoclax) IP 3 times a week
8 mice-Nerofe IP 3 times a week+ABT-263 (navitoclax) IP 3 times a week
8 mice-Nerofe IP 3 times a week+GX15-070 (obatoclax) IP 3 times a week
8 mice-Nerofe IP 3 times a week+A-1331852 (obatoclax) IP 3 times a week. Weight of mice is measured twice a week and tumor volume is also measured twice a week.

1.-36. (canceled)

37. A combined composition comprising at least one inhibitor of a B-cell lymphoma 2 (Bcl2) prosurvival protein and an isolated peptide comprising the amino acid sequence denoted by SEQ ID NO. 1 or any functional derivative thereof, or pharmaceutically acceptable salt thereof and at least one pharmaceutically acceptable carrier, diluent, excipient and/or additive.

38. The composition of claim 37, wherein said modified isolated peptide has at least 70%, or 80%, or 90%, or 95% identity to the corresponding sequence of SEQ ID NO: 1.

39. The composition of claim 37, wherein said Bcl-2 prosurvival protein is at least one of Bcl-2, B-cell lymphoma-extra large (Bcl-XL), Myeloid Cell Leukemia-1 (Mcl-1), B-cell lymphoma-w (Bcl-w), B-cell lymphoma 2 related protein A1 (Bcl2A1) and B-cell lymphoma 2 like 10 (Bcl-B/Bcl2L10).

40. The composition of claim 37, wherein said at least one inhibitor of a Bcl2 prosurvival protein is at least one Bcl-2 homology 3 (BH3) mimetic compound, optionally wherein said BH3 mimetic compound is at least one of 4-{4-[(4′-Chloro[1,1′-biphenyl]-2-yl)methyl]piperazin-1-yl}-N-(4-{[(2R)-4-(dimethylamino)-1-(phenylsulfanyl) butan-2-yl]ami-no}-3-nitrobenzene-1-sulfonyl)benzamide (ABT-737), 4-(4-{[2-(4-Chlorophenyl)-4,4-dimethyl-1-cyclohexen-1-yl]methyl}-1-piperazinyl)-N-({3-nitro-4-[(tetrahydro-2H-pyran-4-ylmethyl)-amino]phenyl}sulfonyl)-2-(1H-pyrrolo[2,3-b]-pyridin-5-yloxy)benzamide (ABT-199), 4-(4-{[2-(4-Chlorophenyl)-5,5-dimethylcyclohex-1-en-1-yl]methyl}piperazin-1-yl)-N-(4-{[(2R)-4-(morpholin-4-yl)-1-(phe-nylsulfanyl) butan-2-yl]amino}-3-(trifluoromethanesulfonyl)benzene-1-sulfonyl)-benzamide (ABT-263), 2-(2-((3,5-Dimethyl-1H-pyrrol-2-yl)methylene)-3-methoxy-2H-pyrrol-5-yl)-1H-indole (GX15-070), or 3-[1-(1-adamantylmethyl)-5-methylpyrazol-4-yl]-6-[8-(1,3-benzothiazol-2-ylcarbamoyl)-3,4-dihydro-1H-isoquinolin-2-yl]pyridine-2-carboxylic acid (A-1331852), or any combinations thereof.

41. The composition of claim 40, wherein said at least one inhibitor of a Bcl2 prosurvival protein is 4-{4-[(4′-Chloro[1,1′-biphenyl]-2-yl)methyl]piperazin-1-yl}-N-(4-{[(2R)-4-(dimethylamino)-1-(phenylsulfanyl) butan-2-yl]ami-no}-3-nitrobenzene-1-sulfonyl)-benzamide (ABT-737), or any a pharmaceutically acceptable salt or hydrate thereof or any stereoisomer or salt thereof.

42. A method of treating, preventing, ameliorating, reducing or delaying the onset of at least one proliferative disorder in a subject in need thereof, comprising the step of administering a therapeutically effective amount of at least one inhibitor of a Bcl2 prosurvival protein and an isolated peptide comprising the amino acid sequence denoted by SEQ ID NO. 1 or any functional derivative thereof, or pharmaceutically acceptable salt thereof, or any compositions, kits or combinations thereof.

43. The method of claim 42, wherein said modified isolated peptide has at least 70%, or 80%, or 90%, or 95% identity to the corresponding sequence of SEQ ID NO: 1.

44. The method of claim 42, wherein said Bcl-2 prosurvival protein is at least one of Bcl-2, Bcl-XL, Mcl-1, Bcl-w, Bcl2A1 and Bcl-B/Bcl2L10.

45. The method of claim 42, wherein said at least one inhibitor of a Bcl2 prosurvival protein is at least one BH3 mimetic compound, optionally wherein said BH3 mimetic compound is at least one of 4-{4-[(4′-Chloro[1,1′-biphenyl]-2-yl)methyl]piperazin-1-yl}-N-(4-{[(2R)-4-(dimethylamino)-1-(phenylsulfanyl) butan-2-yl]ami-no}-3-nitrobenzene-1-sulfonyl)benzamide (ABT-737), 4-(4-{[2-(4-Chlorophenyl)-4,4-dimethyl-1-cyclohexen-1-yl]methyl}-1-piperazinyl)-N-({3-nitro-4-[(tetrahydro-2H-pyran-4-ylmethyl)-amino]phenyl}sulfonyl)-2-(1H-pyrrolo[2,3-b]-pyridin-5-yloxy)benzamide (ABT-199), 4-(4-{[2-(4-Chlorophenyl)-5,5-dimethylcyclohex-1-en-1-yl]methyl}piperazin-1-yl)-N-(4-{[(2R)-4-(morpholin-4-yl)-1-(phe-nylsulfanyl) butan-2-yl]amino}-3-(trifluoromethanesulfonyl)benzene-1-sulfonyl)-benzamide (ABT-263), 2-(2-((3,5-Dimethyl-1H-pyrrol-2-yl)methylene)-3-methoxy-2H-pyrrol-5-yl)-1H-indole (GX15-070), or 3-[1-(1-adamantylmethyl)-5-methylpyrazol-4-yl]-6-[8-(1,3-benzothiazol-2-ylcarbamoyl)-3,4-dihydro-1H-isoquinolin-2-yl]pyridine-2-carboxylic acid (A-1331852).

46. The method of claim 45, wherein said at least one inhibitor of a Bcl2 prosurvival protein is 4-{4-[(4′-Chloro[1,1′-biphenyl]-2-yl)methyl]piperazin-1-yl}-N-(4-{[(2R)-4-(dimethylamino)-1-(phenylsulfanyl) butan-2-yl]ami-no}-3-nitrobenzene-1-sulfonyl)-benzamide (ABT-737), or any a pharmaceutically acceptable salt or hydrate thereof or any stereoisomer or salt thereof.

47. The method of claim 45, wherein said at least one inhibitor of a Bcl2 prosurvival protein and an isolated peptide comprising the amino acid sequence denoted by SEQ ID NO. 1 are administered concomitantly or consecutively.

48. The method of claim 45, further comprises administering at least one additional anti-cancer agent.

49. The method of claim 45, wherein at least one of:
(i) proliferative disorder is associated with Bcl2 over-expression; and/or
(ii) said proliferative disorder is at least one hematological cancer or neoplasm, optionally said hematological cancer or neoplasm is at least one of chronic lymphocytic leukemia (CLL), acute myeloid leukemia (AML), mutated KRAS tumors, lymphomas, multiple myeloma, blastic plasmacytoid dendritic cell neoplasm, T-cell prolymphocytic leukemia or myelodysplasia.

50. A kit comprising:
(a) at least one inhibitor of a Bcl2 prosurvival protein or any pharmaceutically acceptable salt thereof, optionally, in a first dosage form;
(b) an isolated peptide comprising the amino acid sequence denoted by SEQ ID NO. 1 or any functional derivative thereof, or pharmaceutically acceptable salt thereof, optionally, in a second dosage form.

51. The kit of claim 50, wherein at least one of:
(i) said modified isolated peptide has at least 70%, or 80%, or 90%, or 95% identity to the corresponding sequence of SEQ ID NO: 1; and/or
(ii) said Bcl-2 prosurvival protein is at least one of Bcl-2, Bcl-xL, Mcl-1, Bcl-w, Bcl2A1 and Bcl-B/Bcl2L 10.

52. The kit of claim 50, wherein said at least one inhibitor of a Bcl2 prosurvival protein is at least one BH3 mimetic compound, optionally wherein said BH3 mimetic compound is at least one of 4-{4-[(4′-Chloro[1,1′-biphenyl]-2-yl)methyl]piperazin-1-yl}-N-(4-{[(2R)-4-(dimethylamino)-1-(phenylsulfanyl) butan-2-yl]ami-no}-3-nitrobenzene-1-sulfonyl)benzamide (ABT-737), 4-(4-{[2-(4-Chlorophenyl)-4,4-dimethyl-1-cyclohexen-1-yl]methyl}-1-piperazinyl)-N-({3-nitro-4-[(tetrahydro-2H-pyran-4-ylmethyl)-amino]phenyl}sulfonyl)-2-(1H-pyrrolo[2,3-b]-pyridin-5-yloxy)benzamide (ABT-199), 4-(4-{[2-(4-Chlorophenyl)-5,5-dimethylcyclohex-1-en-1-yl]methyl}piperazin-1-yl)-N-(4-{[(2R)-4-(morpholin-4-yl)-1-(phe-nylsulfanyl) butan-2-yl]amino}-3-(trifluoromethanesulfonyl)benzene-1-sulfonyl)-benzamide (ABT-263), 2-(2-((3,5-Dimethyl-1H-pyrrol-2-yl)methylene)-3-methoxy-2H-pyrrol-5-yl)-1H-indole (GX15-070), or 3-[1-(1-adamantylmethyl)-5-methylpyrazol-4-yl]-6-[8-(1,3-benzothiazol-2-ylcarbamoyl)-3,4-dihydro-1H-isoquinolin-2-yl]pyridine-2-carboxylic acid (A-1331852).

53. The kit of claim 52, wherein said at least one inhibitor of a Bcl2 prosurvival protein is 4-{4-[(4′-Chloro[1,1′-biphenyl]-2-yl)methyl]piperazin-1-yl}-N-(4-{[(2R)-4-(dimethylamino)-1-(phenylsulfanyl) butan-2-yl]ami-no}-3-nitrobenzene-1-sulfonyl)-benzamide (ABT-737), or any a pharmaceutically acceptable salt or hydrate thereof or any stereoisomer or salt thereof.

54. A method of treating, preventing, ameliorating, reducing or delaying the onset of at least one proliferative disorder in a subject treated with at least one inhibitor of a Bcl2 prosurvival protein, the method comprising the step of administering a therapeutically effective amount of an isolated peptide comprising the amino acid sequence denoted by SEQ ID NO. 1 or any functional derivative thereof, or pharmaceutically acceptable salt thereof, or any compositions, kits or combinations thereof, to said subject.

55. A method of treating, preventing, ameliorating, reducing or delaying the onset of at least one proliferative disorder in a subject treated with at least one isolated peptide comprising the amino acid sequence denoted by SEQ ID NO. 1, the method comprising the step of administering a therapeutically effective amount of at least one inhibitor of a Bcl2 prosurvival protein or any functional derivative thereof, or pharmaceutically acceptable salt thereof, or any compositions, kits or combinations thereof to said subject.
